(19) United States
(12) Patent Application Publication
(10) Pub. No.: US 2013/0302278 A1
(43) Pub. Date: Nov. 14, 2013

(54) USE OF TLR AGONISTS AND/OR TYPE 1 INTERFERONS TO ALLEVIATE TOXICITY OF TNF-R AGONIST THERAPEUTIC REGIMENS

(71) Applicant: Immurx Inc., Lebanon, NH (US)

(72) Inventors: Randolph J. Noelle, Plainfiled, NH (US); Ross M. Kedl, Centennial, CO (US); Cory L. Ahonen, Enfield, NH (US)

(21) Appl. No.: 13/863,460

(22) Filed: Apr. 16, 2013

Related U.S. Application Data
(63) Continuation of application No. 12/664,921, filed on Apr. 13, 2011, now abandoned.
(60) Provisional application No. 60/944,288, filed on Jun. 15, 2007.

(51) Int. Cl. A61K 39/395; A61K 38/21; A61K 39/00; A61K 45/06; A61K 39/02; A61K 39/002; A61K 35/74; A61K 36/06; A61K 38/19; A61K 39/12

(52) U.S. Cl. CPC A61K 39/3955; A61K 38/191; A61K 38/21; A61K 39/00; A61K 39/12; A61K 39/02; A61K 39/002; A61K 39/0002; A61K 39/0011; A61K 35/74; A61K 36/06; A61K 45/06; A61K 39/0005
USPC 424/85.4; 424/85.1; 424/158.1; 424/184.1; 424/204.1; 424/234.1; 424/265.1; 424/274.1; 424/277.1; 424/93.4; 424/93.51; 424/195.16; 424/780

(57) ABSTRACT

Improved (safer and more effective) methods of therapy using TNF-R agonists, e.g., CD40 agonists are provided. These methods provide for the addition of an amount of a type 1 interferon and/or a TLR agonist that is effective to prevent or reduce the toxicity (liver toxicity) that may otherwise result in some patients of the TNF-R agonist is used as a monotherapy (without the type 1 interferon and/or TLR agonist).

### PRIORITY INFORMATION

This application claims benefit of priority to provisional application Ser. No. 60/944,288 filed on Jun. 15, 2007 and further claims priority to and is a continuation-in-part of U.S. Ser. No. 10/748,01 filed on Dec. 30, 2003 which claims priority to U.S. Provisional U.S. Ser. No. 60/437,398 filed on Dec. 30, 2002 and also claims priority to and is a continuation in part of U.S. Ser. No. 11/743,978 filed on May 3, 2007 which in turn claims priority to U.S. Provisional 60/842,009 filed on Sep. 5, 2006; 60/809,821 filed on Jun. 1, 2006 and 60/796,867 filed on May 3, 2006. All of these applications are incorporated by reference in their entirety herein.

### FIELD OF THE INVENTION

The invention generally relates to methods of alleviating toxicity, especially liver toxicity observed upon administration of TNF/TNF-R super family agonists, most especially CD40 agonists, by further administering in a therapeutic or immune adjuvant regimen that comprises the administration of a TNF/TNF-R agonist that causes liver toxicity when used as a monotherapy an amount of at least one type 1 interferon and/or toll-like receptor (TLR) agonist sufficient to prevent or alleviate said toxicity, especially liver toxicity. Also, the addition of the type 1 interferon and/or TLR agonist allows for the TNF-R agonist to be administered at higher dosages thereby enhancing efficacy. These therapeutic regimens include by way of example use of these immune agonist and/or cytokine immunostimulatory combinations for treating various chronic diseases including cancer, infectious diseases, autoimmune diseases, allergic and inflammatory diseases.

### BACKGROUND OF THE INVENTION

The past 10 years have witnessed an exponential growth in the identification of cancer target antigens, a similar pace for the development of human adjuvants to effectively immunize against these targets has lagged. The molecular identification of Toll-like Receptors and their ligands, and receptor-ligands that control adaptive immunity have provided the first logical, hypothesis-based strategies to molecularly concoct adjuvants so as to elicit protective immune responses to cancer. Parallel to the importance of TLRs in mobilizing the innate immune response, CD40 and its ligand are the central activators for the development of the adaptive immune responses.

Perhaps one of the weakest aspects of our approach to fight cancer, is the lack of adjuvants that can elicit robust, long-lasting immunity to cancer-related antigens. In the past, we have relied on the use of agents that appeared to induce inflammation. Alum is salts of aluminum hydroxide and phosphate and primarily elicits humoral-mediated immune responses. This adjuvant was first employed in 1926 and was effectively grandfathered in when the FDA first assumed new drug approval authority in 1938. Alum is the only FDA approved adjuvant, and is a component of a number of our commonly used vaccines, like tentanus toxoid. There are many other adjuvants (non-cytokine) that have been employed in cancer clinical trials like Bacille Calmette-Guérin (BCG), keyhole limpet hemocyanin (KLH), incomplete Freund's adjuvant (IFA), all which have poorly understood mechanisms of action and modest adjuvant activities. Not until 1999 when the first studies elucidating the receptors for immune adjuvants (Toll-like receptors) emerged on the horizon, did a molecular understanding of how these “non-specific” activators of the immune system trigger innate immunity. TLRs are type 1 membrane proteins that are expressed on hematopoietic and non-hematopoietic cells. Currently, there are 11 members in the TLR family. These receptors are characterized by their capacity to recognize pathogen-associated molecular patterns (PAMP) expressed by pathogenic organisms. Typical PAMPS include LPS, DNA (CpG), lipoproteins, ssRNA, and glycolipids. Whether there are true endogenous ligands for TLRs is still controversial, although it has been reported that TLR2 and TLR4 are able to recognize several self-proteins including members of heat shock protein family hsp60 and hsp70.

In general, triggering of TLR elicits profound inflammatory responses through enhanced cytokine production (IL12, IL18, etc), chemokine receptor expression (CCR2, CCR5 and CCR7), and costimulatory molecule expression. As such, these receptors in the innate immune systems exert control over the polarity of the ensuing acquired immune response.

CD154 or CD40L, the ligand for CD40 (CD40L, gp39) is a 32-39 kD member of the Tumor Necrosis Factor Family, which includes TNF-α, lymphotoxin, FasL, CD30L, CD27L, 4-1BBL, and OX-40L. Activated CD4 T-cells are the predominant cell type responsible for CD154 expression. Expression of CD154 on CD8⁺ T-cells, eosinophils, mast cells and basophils, NK cells, and DCs has also been described. The receptor for CD154, CD40 is a member of the tumor necrosis factor receptor (TNF-R) superfamily that includes TNF-RI (p55), TNF-RII (p75), p75 neurotrophin receptor, fas, CD30, CD27, 4-1BB, and OX-40. It is a 50-kDa membrane protein whose tissue distribution was originally thought to be restricted to B cells, DCs (DC's) and basal epithelial cells however, later studies have shown functional expression of CD40 on monocytes/macrophages, microglial cells and endothelial cells.

In vitro studies on isolated DCs have shown that CD40 triggering alters the expression of cytokines (IL12, IL15) chemokines (IP10, MIP-beta MIP-1alpha and IL-8), co-stimulatory molecule expression (CD80, CD86) and chemokine receptors. All of these effects culminate in the ability of CD40-activated DCs to stimulate enhanced T cell proliferation and differentiation. Our own data shows that CD154 exerts far more profound effects on the early signaling, cytokine production and chemokine production compared to TNFalpha and RANKL. One other critical impact of CD40 triggering of DCs is the change in the turnover of peptide-MHCII. Lanzavecchia has shown using LPS and we have shown using, that maturation of DCs with a CD40 agonist facilitates the accumulation of MHCII-peptide complexes on the surface of DCs. Studies from our lab and others, indicate that CD40 appears to be a critical longevity signal for DCs in vivo.

The success of CD40 agonists to elicit CMI in the absence of CD4⁺ T cells generated substantial enthusiasm to use CD40 agonists as adjuvants for cancer vaccines. A series of studies by Glennie and co-workers showed that one can achieve tumor regression of CD40⁺ lymphoma using □CD40, but the doses of anti-CD40 were very high (250 ug/day, days 2-5), and oddly, the tumor inoculum needed for immunization was very high (5×10⁷/mouse). Nonetheless, clinical remission of these CD40⁺ lymphoma was impressive. Less impressive were studies on hematopoietic tumors which were CD40⁻. It is likely that the successes with CD40⁺ lymphomas and leukemias were due to direct effects of CD40 agonists on the tumor. For lymphomas and leukemias, CD40 agonists may also enhance their APC activities, and at the same time enhance their apoptosis. Later studies by this same group, however, did demonstrate that CD40 agonists could exert beneficial therapeutic effects on solid tumors. With solid tumors, a number of studies have shown that CD40 activation promotes apoptotic death and that CD40 expression is an important factor in the generation of tumor-specific T-cell responses that contribute to tumor cell elimination. Other groups, like that of Melief and co-workers have shown that CD40 agonists alone or TLTR agonists alone could elicit effective therapeutic on Ad5E1A expressing (CD40−) tumors in vivo (tumor type not described). Using a renal cell carcinoma model, Murphy and co-workers have shown that only the combination of an agonist anti-CD40 and IL-2, but neither agent administered alone, induced complete regression of metastatic tumor and specific immunity to subsequent rechallenge in the majority of treated mice. At this time efficacy with CD40 agonists alone is unpredictable. It is not clear if CD40 expression on the tumor is important, if tumor burden is important, if CD40 alone is adequate and if there is a distinctive difference in the efficacy of CD40 agonist therapy in liquid or solid tumors.

CD40 is a reasonable target for inducing heightened CMT responses for the purposes of tumor protection, yet the data in the literature suggested that it was not applicable in a wide range of tumors. Those skilled in the art including the inventors have worked intensively to try to develop a general method to enhance protective tumor immunity using anti-CD40 antibody as a monotherapy, and failed. Any and all parameters of dose of antibody, timing, route of inoculation, tumor type, different mabs, etc were extensively tested yet these efforts proved futile, except in B lymphoma and leukemia models, as reported by Glennie.

A recent study from Kedl and co-workers has shed much light on some of the important parameters that may influence the generation of protective CTL when using CD40 agonists. Using tetramer staining for SIINFYKL-specific CTL, and OVA-transduced B16, they showed that anti-CD40 antibody agonists actually accelerated the loss of SIINFYKL-specific CTL. However, if immunization were done with a vaccinia virus carrying a SIINFYKL minigene, enhanced CTL expansion was observed using anti-CD40 antibody agonists. It was concluded that long-term immunization to tumor antigens are only enhanced by CD40 agonists if those tumor antigens are delivered in viral vectors or in the context of inflammation. Hence, the great disparities in the outcome of innumerable tumor models may be due to the inadvertent addition of co-inflammatory mediators that synergize with the antibody CD40 agonist.

Such in vivo studies led to a number of recent reports on the requirements of co-signals for the activation of DCs by CD40 agonists. Published studies, show that CD40 engagement alone is insufficient to induce IL12p70 production by DCs in vitro and in vivo. By evaluating mRNA for p40 and p35, the present inventors showed that co-engagement via TLR (STAg, an extract from Toxoplasma gondi) and CD40 is critical for enhanced p35 mRNA expression and the production of IL12p70. This study was followed by an investigation using human DCs where it was shown that CpG DNA was a critical co-stimulus with CD40 signaling for IL12p70 production in vitro. Taken together, these were the first studies to document that CD40 was necessary but not sufficient to drive DC certain aspects of DC maturation. However, they did not provide compelling evidence that the combined actions of CD40 and TLR agonism was essential to fulminately elicit CMI.

To increase the effectiveness of an adaptive immune response, such as in a vaccination protocol or during a microbial infection or cancer, it is therefore important to develop novel, more effective, vaccine adjuvants but which do not elicit adverse toxic side effects. The present invention satisfies this need and provides other advantages as well.

### SUMMARY OF THE INVENTION

This invention relates to improved therapies involving the administration of immune adjuvants comprising the combination of (i) at least one TNF-R agonist, preferably an CD40 agonist comprised in a dosage that in clinical studies when used as a monotherapy elicits liver toxicity in some subjects (ii) an amount of at least one type I interferon and/or at least one TLR agonist, at a dosage which is statistically effective to reduce or eliminate the liver toxicity of said TNF-R agonist dosage if administered as a monotherapy and (iii) optionally an antigen against which a cellular immune response is desirably elicited, e.g., a microbial, viral or tumor antigen. The present invention further relates to the use of such therapies and compositions for use therein as immune adjuvants and for treating conditions wherein T cell immunity is desirably enhanced but without an undesirable elicitation of liver toxicity.

The use of synergistic adjuvants comprising a TLR agonist and a CD40 agonist or-and optionally an antigen is disclosed in U.S. Ser. No. 10/748,010 filed on Dec. 30, 2003 which application is incorporated by reference in its entirety herein. This prior application exemplifies a variety of isolated TLR agonist compounds and their use in conjunction with CD40 and other TNF-R agonists and optionally a desired antigen to which a T cell immune response is desirably to be elicited against and the use thereof as immune adjuvants for treating conditions such as cancer, infection, autoimmune diseases and other conditions wherein antigen specific T cell immunity is desired.

This invention is an extension thereof as it relates to the discovery that type 1 interferons and/or TLR agonists can be used to reduce or eliminate the toxic side effects of TNF-R agonist therapeutic regimens. The subject therapeutic regimen may be administered to a host in need of such treatment as a means of:
(i) generating enhanced (exponentially better) primary and memory CD8+ T cell responses relative to immunization with either agonist alone;
(ii) inducing the exponential expansion of antigen-specific CD8+ T cells, and
(iii) generating protective immunity even in CD4 deficient or depleted hosts and
(iv) generates said therapeutic responses while eliciting substantially less liver toxicity than if said TNF-R agonist were used as a monotherapy

In contrast to some previous TNF-R agonist therapeutic regimens, the present regimen is both safe and effective, i.e., it does not appreciably result in any toxicity to the liver. Thereby, the present invention provides for enhanced efficacy as the TNF-R agonist, e.g., a CD40 agonist may be used at higher dosages, e.g. 2-fold to even 10-fold higher than present therapeutic regimens, but without liver toxicity. This will enhance the efficacy thereof against target cells, e.g. virally infected or tumor cells.

The present invention in particular reveals the impact of combination therapy with that of monotherapy on the antigen-specific immune responses to melanoma at the cellular and molecular levels and on toxicity. The studies contained in the examples infra demonstrate the profound utility of CD40 and TLR agonists when combined in an adjuvant platform in a murine model of cancer. The data show that vaccination induces extremely high frequencies of primary and memory self-reactive CD8⁺ T cells that infiltrate metastatic target organs and control tumor growth. Combination therapy also reduces the ratio of regulatory T cells (T^{regs}) to CD8⁺ T cells at the tumor site and allows persistent effector CD8⁺ T-cell function. Finally, the overt hepatotoxicity induced by CD40 monotherapy is ablated by combination therapy. These studies show that combinatorial use of CD40 and TLR agonists provides greater therapeutic efficacy with limited toxicity and provides the principles on which to build new multifactorial adjuvants for use in clinical trials.

Based on the results infra, these immune adjuvant combinations which optionally may further include an antigen may be used in treating any disease or condition wherein the above-identified enhanced cellular immune responses are therapeutically desirable, especially infectious diseases, proliferative disorders such as cancer, allergy, autoimmune disorders, inflammatory disorders, and other chronic diseases wherein enhanced cellular immunity is a desired therapeutic outcome. Preferred applications of the invention include especially the treatment of infectious disorders such as HIV infection and cancer.

### DETAILED DESCRIPTION OF THE FIGURES

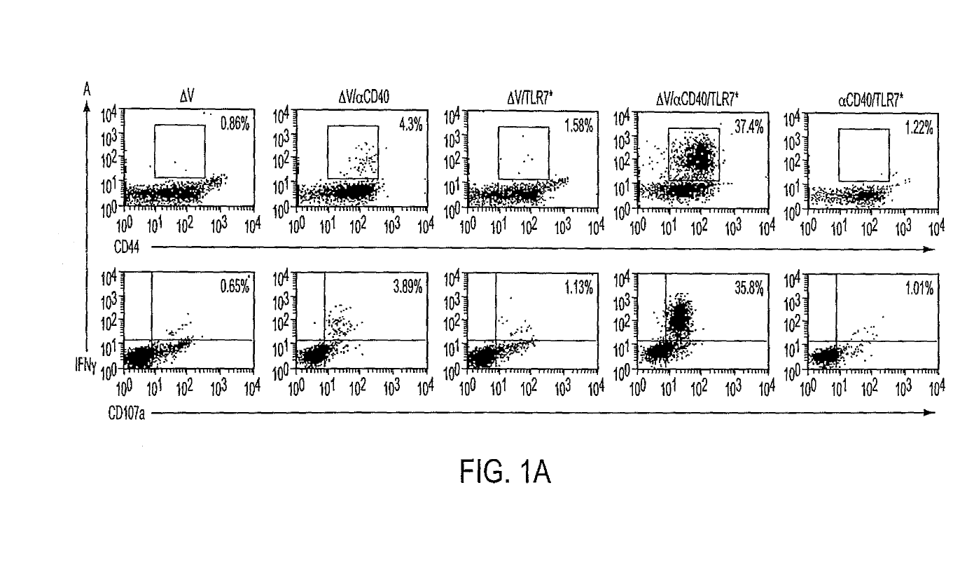

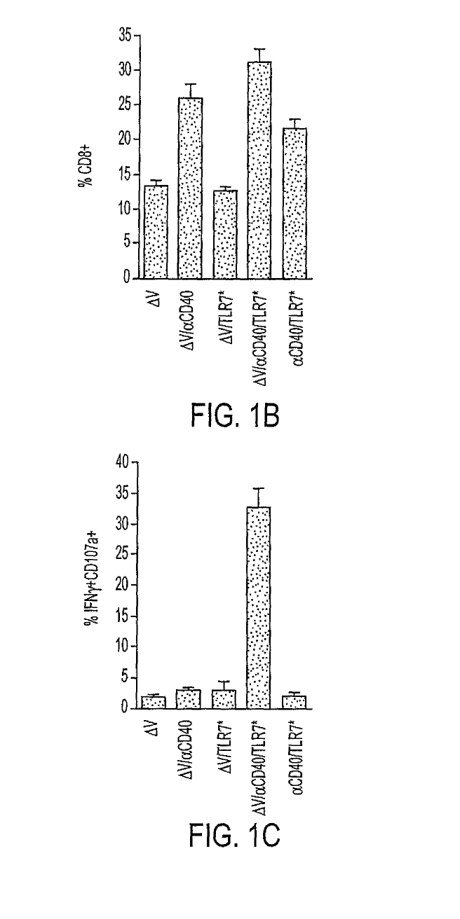

FIG. 1 This Figure contains experiments that show that concomitant signaling through CD40 and TLR7 drives the expansion of self-antigen specific CD8+ T cells with enhanced cytolytic activity. In the experiments therein C57BL/6 mice were immunized intravenously with 100 μg of the tumor-associated antigen V, 100 μg CD40 FGK45, and 100 μg S-27609 in combinations as indicated. Seven days later, mice were bled and cells were restimulated in vitro with TRP2₍₁₈₀₋₁₈₈₎ to assess the ability to produce IFN and translocate CD107a as described in “Methods.” Lymphocytes were identified by forward and side scatter and subsequently gated on all CD8⁺ events. (A) Representative dot plots from vaccinated mice. The numbers in the upper right corners indicate the frequency of CD8⁺ T cells that are positive for IFN and CD44 (top row) or IFN and CD107a (bottom row). (B) Percentage of peripheral blood lymphocytes expressing the CD8 antigen. P.001 by one-tailed ANOVA (C) Quantification of the percentages of CD8⁺ cells that degranulated in response to peptide restimulation. In all cases, data presented are representative of at least 3 independent experiments. Data are plotted as means plus or minus SEM (n=8 in each group). P.001 by one-tailed ANOVA.

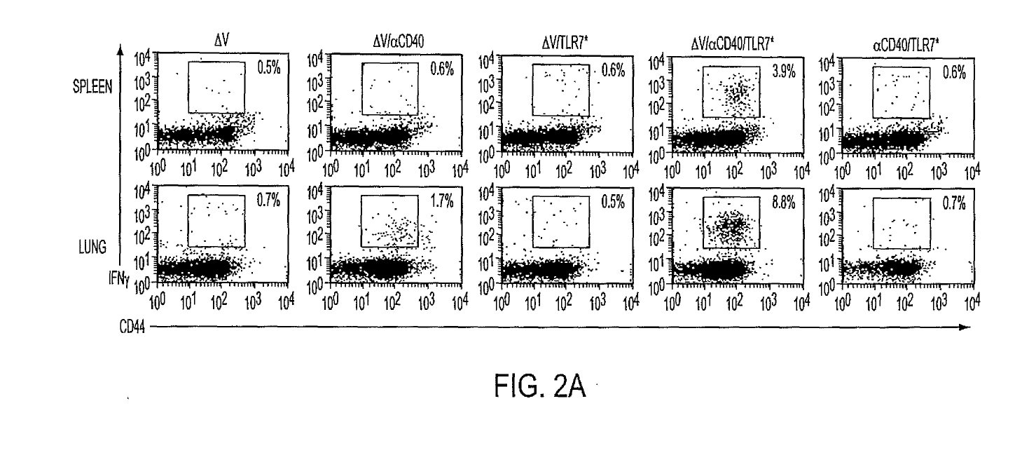

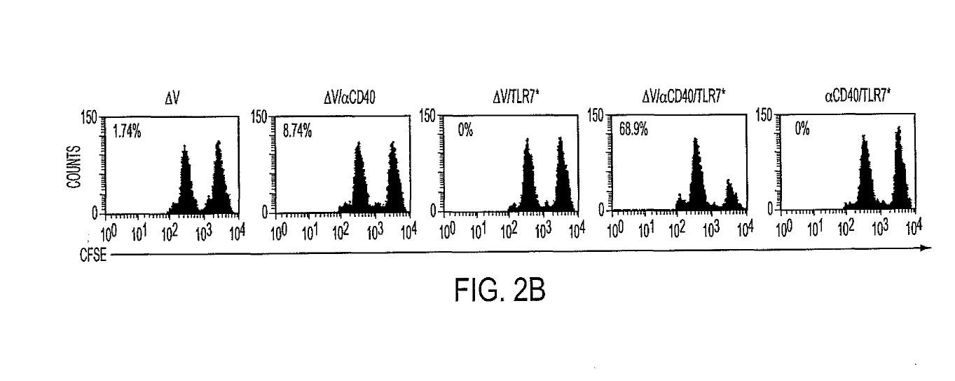

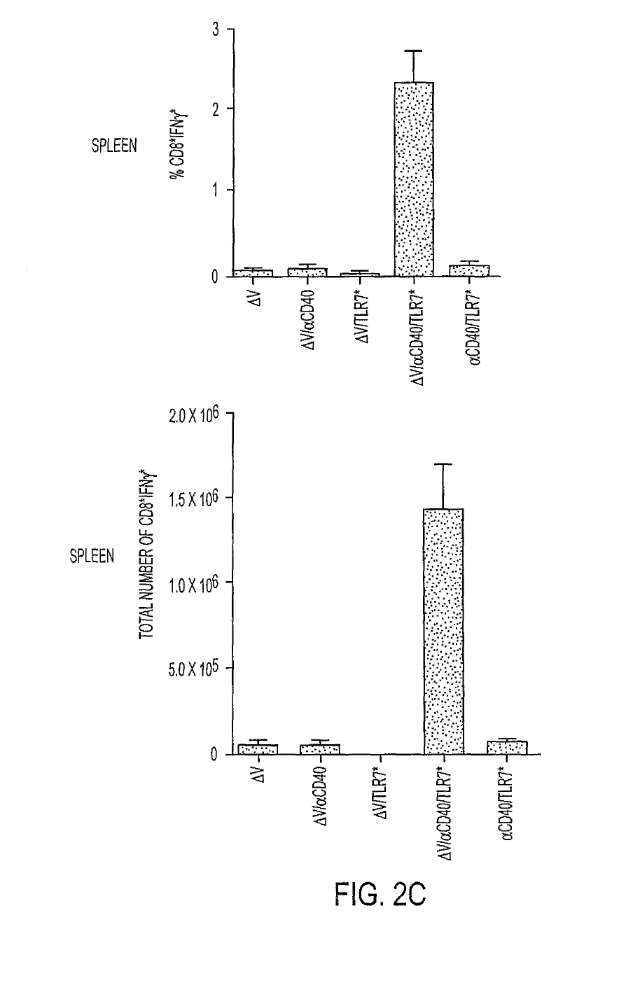

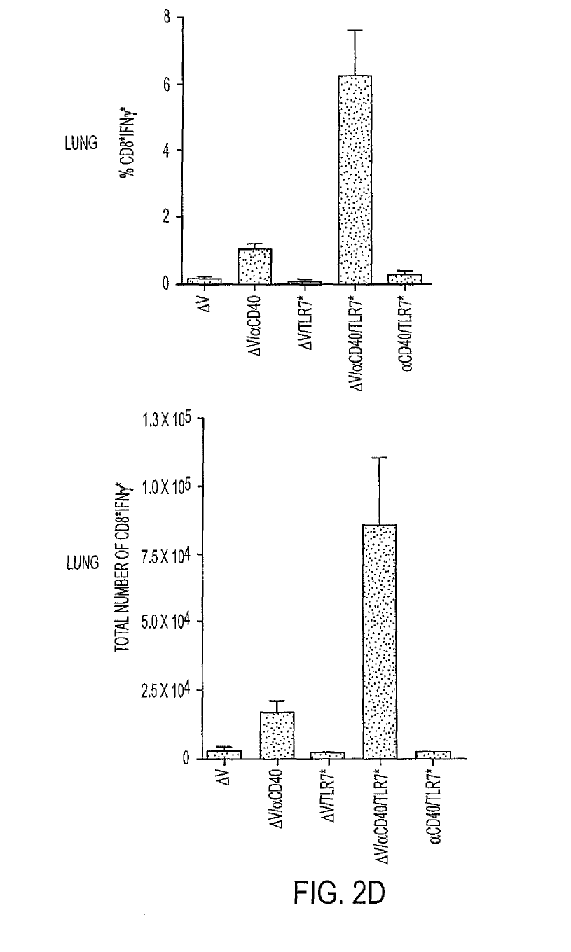

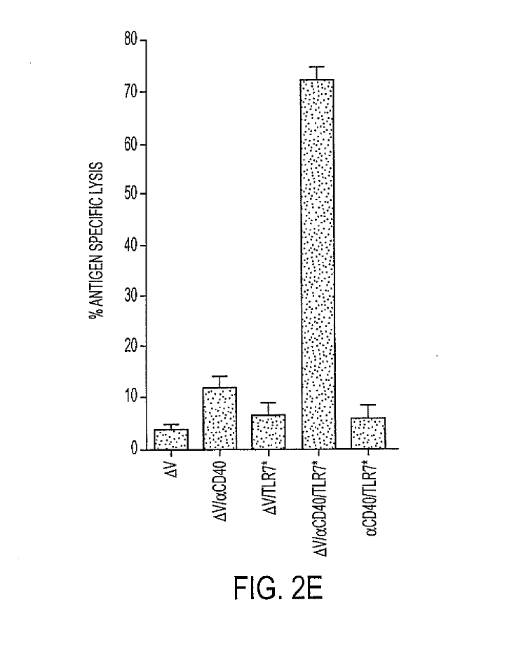

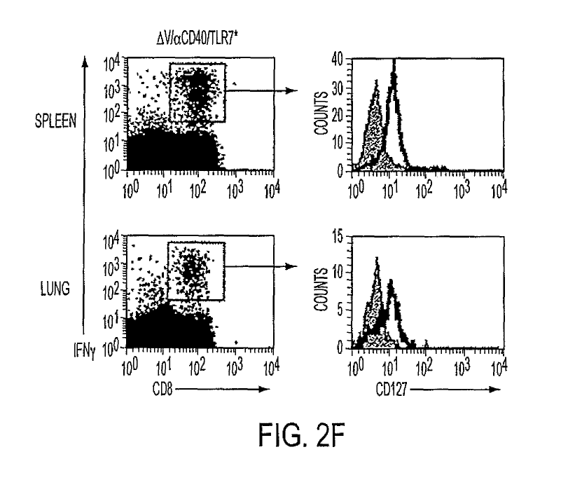

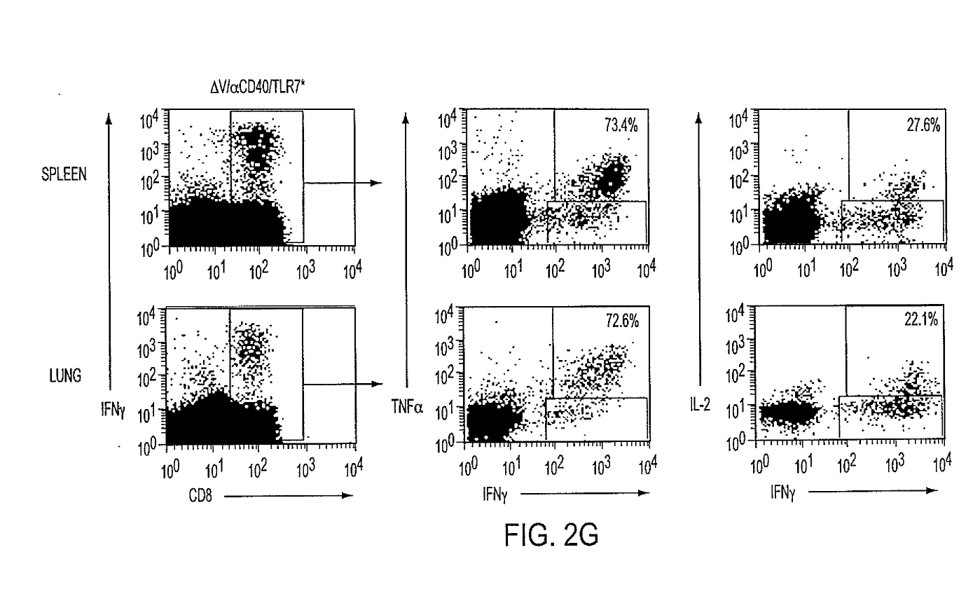

FIG. 2 This figure contains experiments which show that in contrast to CD40 agonist monotherapy, CD40 agonist/TLR6 agonist therapy rescues T cell function. In the experiments depicted in FIG. 2, mice were immunized with 100 μg each of V peptide, CD40, and S-27609 in combinations as indicated. Memory CD8S functionality was assessed 65 days later. (A) Representative dot plots of IFN secretion by memory CD8⁺ T cells isolated from spleens and lungs of vaccinated mice. Dot plots are gated on live CD8⁺ cells, and numbers indicate the percentage of cells positive for both IFN and CD44. (B) Memory CD8⁺ T-cell cytolytic activity was assessed by performing an in vivo cytotoxicity assay. Numbers reflect the percentage of antigen-specific lysis. (C,D) Quantification of relative and absolute numbers of memory CD8 cells expressing IFN in the spleen (C) and lung (D). Absolute numbers of positive cells were determined by multiplying the relative percentage of each cell population by the total number of cells isolated from each tissue. (E) Quantification of the in vivo cytotoxicity assay presented in panel B. P.001 by one-tailed ANOVA. (F) CD127 expression on IFN⁺-memory CD8⁺ T cells derived from spleens or lungs of vaccinated mice. Isotype controls are shown as filled histograms. (G) Cytokine production by memory CD8⁺ T cells. Cells from panel F were analyzed for the ability to produce TNF and IL-2. Numbers reflect the percentage of CD8⁺IFN⁺ cells that also are positive for TNF or IL-2. In all cases, data are pooled from at least 2 independent experiments with 4 or more mice/group per experiment and plotted as means (±SEM).

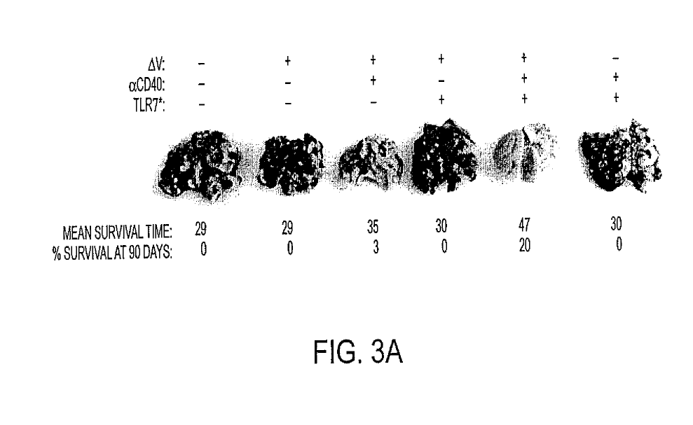

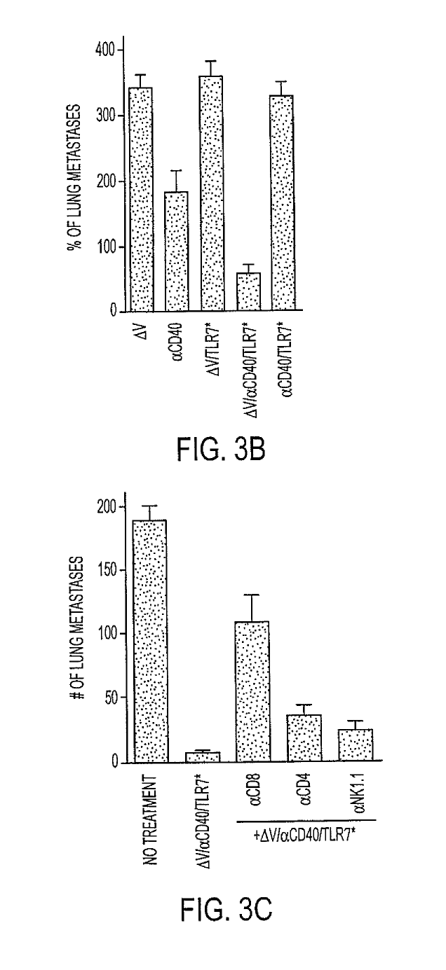

FIG. 3 This figure contains experiments that shoe that anti-CD40/TLR7 agonist therapeutic intervention slows the progression of metastatic melanoma. Therein, C57BL/6 mice were challenged with 10⁵ metastatic B16.F10 melanoma cells intravenously. Four days later, mice were vaccinated with 100 μg of the tumor-associated antigen V, 100 μg CD40 FGK45, and 100 μg S-27609 in combinations as indicated. After 24 days, mice were killed, lungs were removed, and metastatic surface tumor nodules were enumerated with the aid of a dissecting microscope. (A) Photograph of macroscopically visible tumor nodules on lungs of mice, 24 days after tumor challenge. Numbers below the lungs reflect the mean survival time and long-term survival rate of mice monitored for therapeutic efficacy. Data are pooled from 3 to 4 independent experiments with greater than 8 mice per group in each experiment. (B) Enumeration of lung metastases. Data are pooled from 2 independent experiments and are presented as means plus or minus SEM (n=16 mice in each group). Data are representative of more than 4 separate experiments with at least 6 mice in each group. (C) Enumeration of lung metastases after effector cell depletion. Mice were treated as above except for the depletion of effector cell populations prior to tumor challenge as described in “Methods.” The data are expressed as means plus or minus SEM (n=8 mice in each group) and are representative of 3 independent experiments

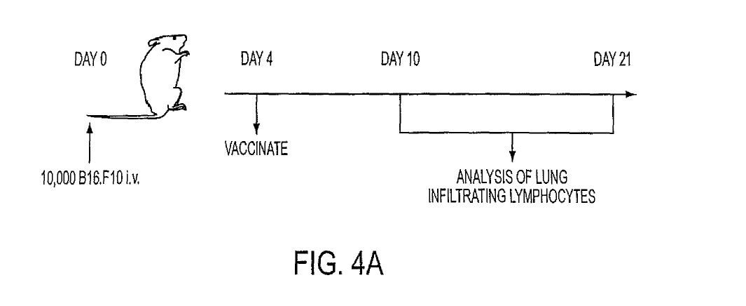

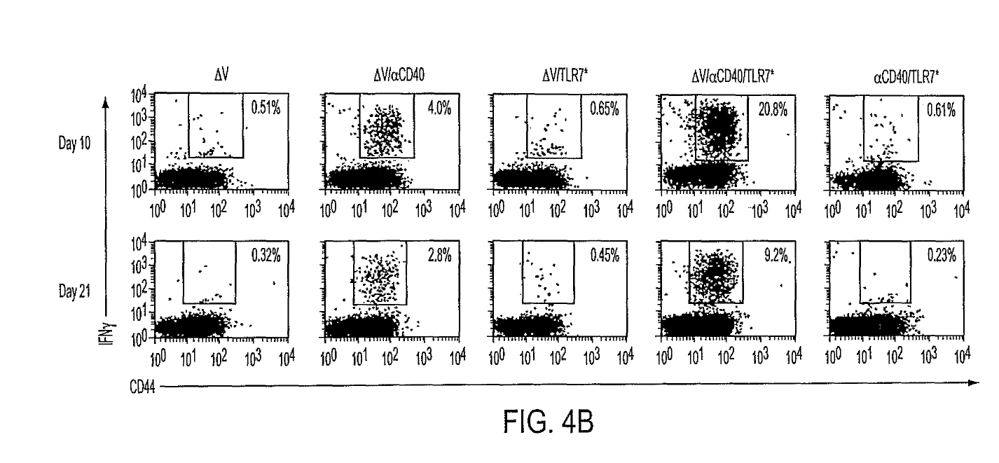

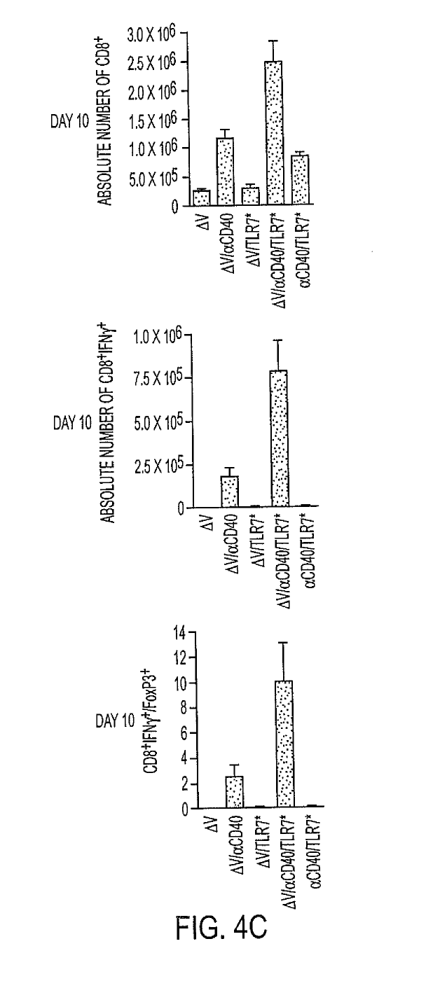

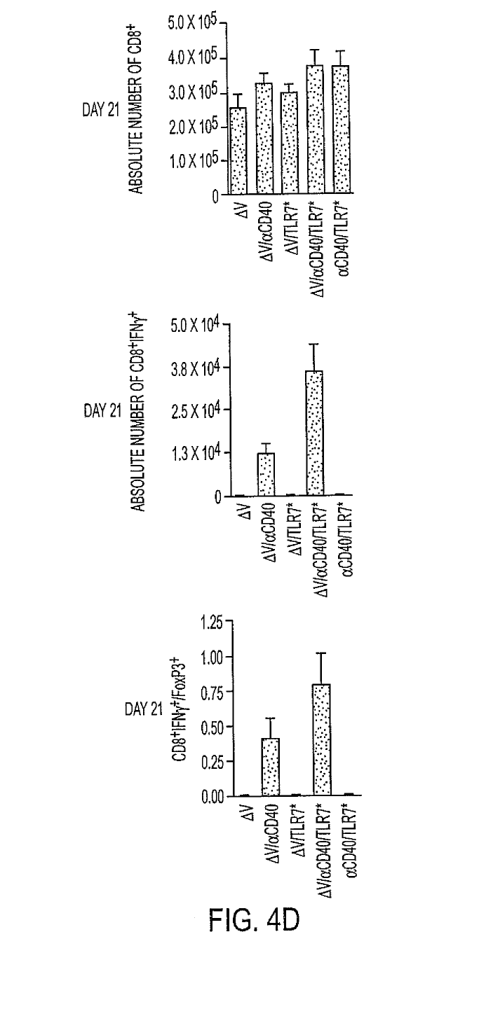

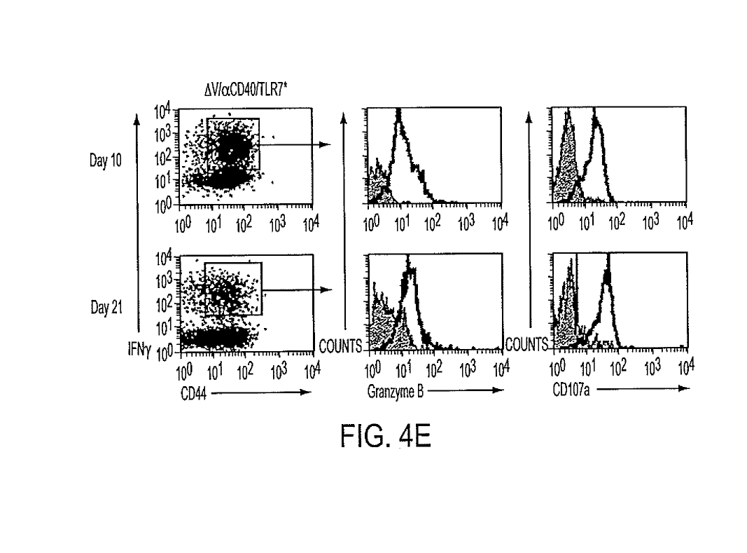

FIG. 4: This figure contains experiments relating to kinetic analysis of infiltrating lymphocytes. Shown in FIG. 4(A) is the experimental design and FIG. 4(B) contains representative dot plots of lymphocytes isolated from metastatic target organs at day 10 or 21 after tumor challenge. Cells were isolated from tumor-bearing lungs as described in “Methods” and subjected to an in vitro restimulation with tumor peptide. Plots are gated on live, CD8⁺ cells. Numbers in the upper right-hand quadrant reflect the frequency of CD8⁺ T cells that are positive for both IFN and the activation marker CD44. Data are representative of 3 independent experiments with 4 mice per group in each experiment. (C,D) Quantification of lung infiltrates at either 10 (C) or 21 (D) days after tumor challenge. Data are plotted as means (±SEM) and represent pooled data from either 2 (C, n=8 mice/group) or 3 (D, n=12 mice/group) independent experiments, with 4 mice/group in each experiment. (E) Effector phenotype of CD8⁺ T cells isolated from lungs of mice vaccinated with tumor antigen plus CD40/TLR7* at either 10 or 21 days following tumor inoculation. The dot plots are first gated on live CD8⁺ cells and then further gated on IFN⁺CD44⁺ populations. Data are representative of at least 2 independent experiments, with 4 mice/group in each experiment.

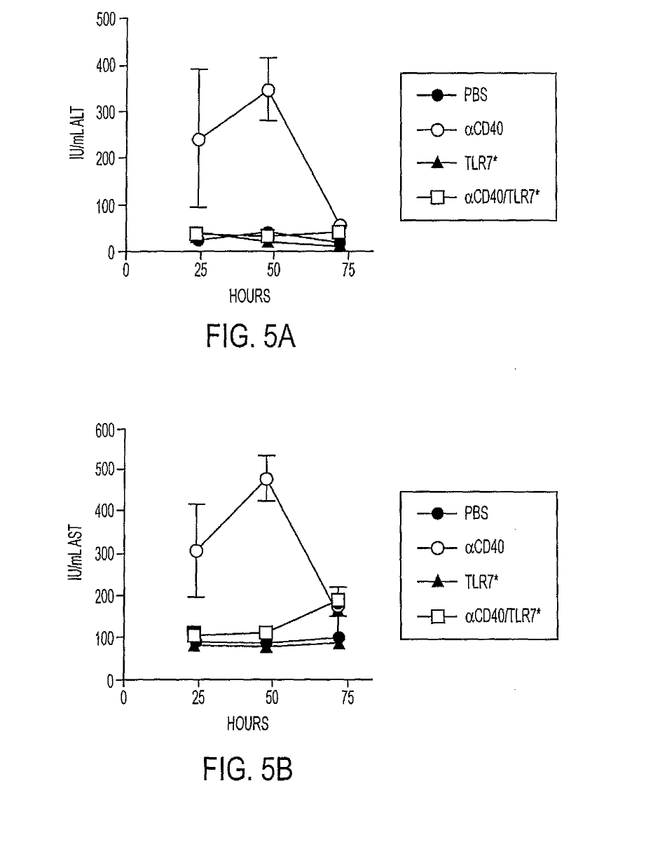

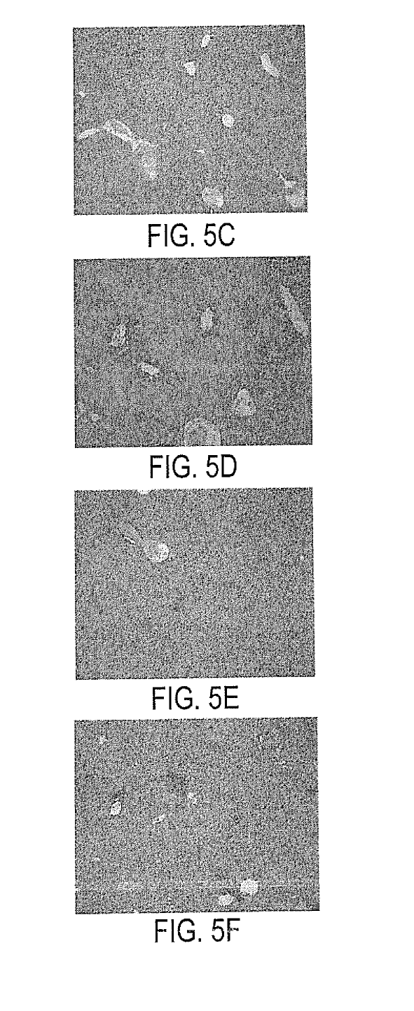

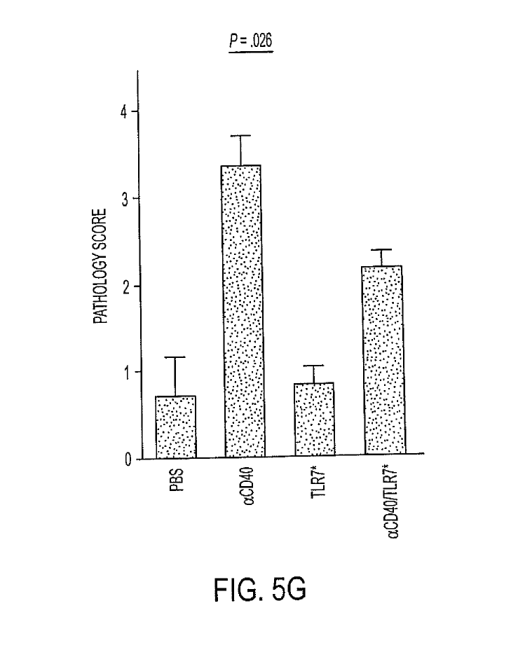

FIG. 5 This figure contains experiments that reveal that the hepatic toxicity associated with CD40 monotherapy is reversed with TLR7 agonism. FIG. 5(A, B) contain kinetic analysis of serum transaminases. Mice were treated with PBS, 100 μg CD40, 100 μg TLR7*, or both intravenously. Serum was isolated at various time points afterward, and serum levels of alanine transaminase (A) or aspartate transaminase (B) were measured as described. Data are representative of 3 independent experiments, with n=3 to 8 mice per group, per time point. (C-F) Histologic analysis of livers treated with PBS (C), 100 kg CD40 (D), 100 μg TLR7* (E), or 100 μg CD40 and 100 μg TLR7*(F) for 48 hours. (G) Semiquantitative assessment of histopathologic changes in livers from mice treated as above for 48 hours. Data are pooled from 2 independent experiments, with n=6 mice in each treatment group. P=0.026 by Mann-Whitney nonparametric test.

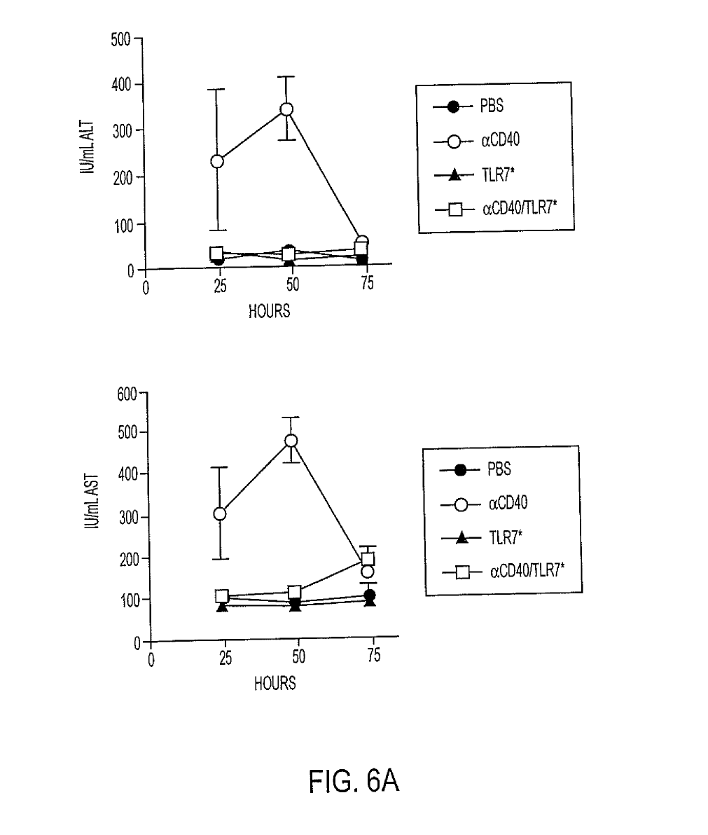

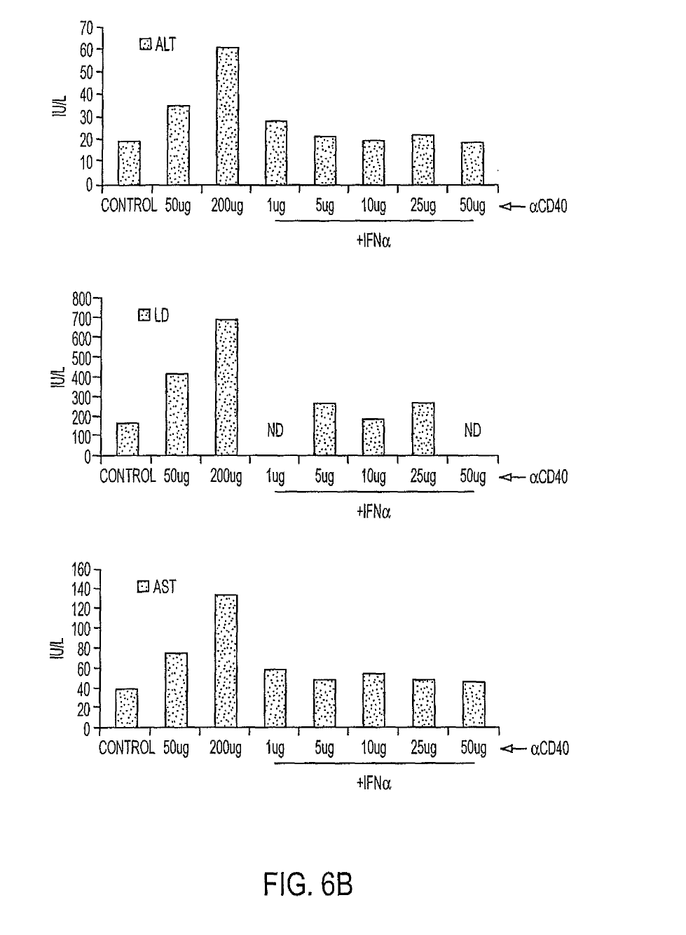

FIG. 6: This figure consisting of FIGS. 6(A) and 6(B) contains additional experiments showing the abatement of liver toxicity by co-administration of a TLR agonist or a type 1 interferon (alpha interferon) with a anti-CD40 antibody agonist. In the experiments therein, hepatocellular injury was biochemically assessed by measuring serum liver enzyme activity. Specifically, mice received 100 mg anti-CD40, 100 mg S-27609 or both i.v. In some cases, mice also received graded doses of recombinant Interferon-alpha (normally, one million international units per mouse). Serum was harvested 24-72 hours later and sent to Charles River Laboratories (Worcester, Mass.) for liver chemistry profile analysis. Alternatively, serum samples were analyzed by the National Jewish Medical Center Core Lab (Denver, Colo.).

### DETAILED DESCRIPTION OF THE INVENTION

The present invention provides a novel methods for alleviating or preventing toxicity, particularly liver toxicity, that is elicited by some therapies involving the administration of TNF/TNF-R agonists, e.g., liver toxicity associated with the administration of some CD40 agonists including CD40 agonistic antibodies and soluble CD40L polypeptides. It has been surprisingly discovered that such toxicity is alleviated or prevented if such therapeutic regimens further include the administration of an amount of a type 1 interferon and/or a TLR agonist sufficient to alleviate or prevent toxicity. Therefore, the present invention reduces the adverse side effects of such therapies, as well as potentially enhancing the efficacy of such therapies as larger dosages of the TNF/TNF-R agonist, e.g., a CD40 agonist may be administered without the danger of eliciting an adverse hepatic reaction in a patient whose liver function may already be compromised because of disease. The subject invention in particular provides for improved (safer and more effective) methods of treating cancer, infectious diseases, autoimmune and inflammatory diseases using a TNF/TNF-R agonist in conjunction with an amount of type 1 interferon and/or TLR agonist sufficient to reduce or prevent liver toxicity that might otherwise result at the administered dosage of TNF/TNF-R agonist.

With respect to the foregoing, while the past 10 years have witnessed an exponential growth in the identification of cancer target antigens, a similar pace for the development of human adjuvants to effectively immunize against these targets has lagged. The molecular identification of Toll-like Receptors and their ligands, and receptor-ligands that control adaptive immunity have provided the first logical, hypothesis-based strategies to molecularly concoct adjuvants so as to elicit protective immune responses to cancer. Parallel to the importance of TLRs in mobilizing the innate immune response, CD40 and its ligand are the central activators for the development of the adaptive immune responses. The data herein show that the use of well-defined agonists that activate specific TLRs, combined with the use of agonists for CD40, elicit profound cell-mediated immune responses to defined peptides that meet or exceed that which is seen with the most potent viral vectors and furthermore reduce or eliminate liver toxicity.

As discussed supra, CD40 is a reasonable target for inducing heightened CMI responses for the purposes of tumor protection, yet the data in the literature suggested that it was not applicable in a wide range of tumors. The inventor's laboratory has worked intensively for a number of years to try to develop a general method to enhance protective tumor immunity using an agonistic anti-CD40 antibody as a monotherapy, and failed. Any and all parameters of dose of antibody, timing, route of inoculation, tumor type, different mabs, etc were extensively tested yet these efforts proved futile, except in B lymphoma and leukemia models, as reported by Glennie.

CD40 Associated Toxicity.

Studies in both mouse and human have shown that the administration of CD40 agonists alone induce toxicity. In intact mice, it has been shown that CD40 agonists induce liver toxicity. In immune deficient mice and non-lethally-irradiated mice, the administration of CD40 agonists induce lethality.

As shown infra, during the course of the inventors' studies with combined administration of CD40 and TLR agonists (or IFNa) it was discovered that the addition of either a TLR agonist or IFNa in vivo to mice treated with a CD40 agonist resolved toxicity. Thus the co-administration of an IFNa and/or a TLR agonist with a CD40 agonist (or other TNF-R agonist that causes similar toxicity if used as a monotherapy) should resolve the toxicity observed in the clinical use of CD40 agonists and other TNF/TNF-R agonists that elicit toxic side effects, especially liver toxicity. That liver toxicity is eliminated or minimized is shown by the examples infra and the supporting Figures containing the data discussed therein.

Therefore, in general this invention comprises improved (safer) therapeutic regimens involving the administration of at least one TNF/TNF-R agonist at a dosage that has been shown to elicit liver toxicity in some subjects at the requisite or desired therapeutic dosage, by the further administration of an amount of at least one type in interferon and/or at least one TLR agonist that is sufficient to reduce or eliminate potential liver toxicity elicited by the TNF/TNF-R agonist of administered as a monotherapy.

Prior to discussing the invention in greater detail the following definitions are provided. Otherwise, the technical terms herein are to be construed as they would be by one skilled in the relevant art.

As used herein, the following terms shall have the meanings set forth:

“Agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors (e.g., a TLR agonist or a TNF/R agonist).

“Antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with immune cells.

“Co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

“Immunostimulatory combination” refers to any combination of components that can be co-administered to provide a therapeutic and/or prophylactic immunostimulatory effect. The components of an immunostimulatory combination can include, but are not limited to, TLR agonists, TNF/R agonists, type 1 interferons, antigens, adjuvants, and the like.

“Mixture” refers to any mixture, aqueous or non-aqueous solution, suspension, emulsion, gel, cream, or the like, that contains two or more components. The components may be, for example, two immunostimulatory components that, together, provide an immunostimulatory combination. The immunostimulatory components may be any combination of one or more antigens, one or more adjuvants, or both. For example, a mixture may include two adjuvants so that the mixture forms an adjuvant combination. Alternatively, a mixture may include an adjuvant combination and an antigen so that the mixture forms a vaccine.

“Synergy” and variations thereof refer to activity (e.g., immunostimulatory activity) of administering a combination of compounds that is greater than the additive activity of the compounds If administered individually.

“TLR” generally refers to any Toll-like receptor of any species of organism. These include TLR1, TLR2, TLR3, TLR4, TLR5, TLR6, TLR7, TLR8, TLR10, TLR TLR0 and TLR11. A specific TLR may be identified with additional reference to species of origin (e.g., human, murine, etc.), a particular receptor (e.g., TLR6, TLR7, TLR8, etc.), or both.

“TLR agonist” refers to a compound that acts as an agonist of a TLR. This includes TLR1, TLR2, TLR3, TLR4, TLR5, TLR6, TLR7, TLR8, TLR9, TLR10, and TLR11 agonists or a combination thereof. Unless otherwise indicated, reference to a TLR agonist compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers. Also, a compound may be identified as an agonist of one or more particular TLRs (e.g., a TLR7 agonist, a TLR8 agonist, or a TLR7/8 agonist). In some embodiments the TLR agonist will comprise a whole virus or microorganism which may be engineered to express a desired antigen. In some embodiments the microorganism or virus which functions as a TLR agonist may be genetically engineered to express a CD40 agonist or another TNF/TNF-R agonist, e.g., a 4-1BB agonist and/or a desired antigen thereby providing the TNF/TNF-R agonist, e.g., CD40 or 4-1BB agonist, TLR agonist and optional antigen in a single microbial or viral vehicle thereby facilitating administration to a host having a condition wherein enhanced antigen specific cellular immune response are desirably elicited. The TLR agonism for a particular compound may be assessed in any suitable manner. For example, assays for detecting TLR agonism of test compounds are described, for example, in U.S. Provisional Patent Application Ser. No. 60/432,650, filed Dec. 11, 2002, and recombinant cell lines suitable for use in such assays are described, for example, in U.S. Provisional Patent Application Ser. No. 60/432,651, filed Dec. 11, 2002 incorporated by reference herein

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for multiple TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NF.kappa.B activation) when the compound is provided at a concentration of, for example, from about 1 .mu·M to about 10 .mu·M for identifying a compound as an agonist of the TLR transfected into the cell. However, different thresholds and/or different concentration ranges may be suitable in certain circumstances. Also, different thresholds may be appropriate for different assays.

In certain embodiments, the TLR agonist can be a natural agonist of a TLR or a synthetic IRM compound. IRM compounds include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-.alpha., IL-1, IL-6, IL-8, IL-10, IL-12, MIP-1, and/or MCP-1. As another example, certain IRM compounds can inhibit production and secretion of certain TH2 cytokines, such as IL-4 and IL-5. Additionally, some IRM compounds are said to suppress IL-1 and TNF (U.S. Pat. No. 6,518,265).

Certain IRMs that are useful as TLR agonists in immunostimulatory combinations of the invention are small organic molecules (e.g., molecular weight less than about 1000 Daltons, and less than about 500 Daltons in some cases), as opposed to large biological molecules such as proteins, peptides, and the like. Certain small molecule IRM compounds are disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,545,016; 6,545,017; 6,558,951; and 6,573,273; European Patent 0 394 026; U.S. Patent Publication No. 2002/0055517; and International Patent Publication Nos. WO 01/74343; WO 02/46188; WO 02/46189; WO 02/46190; WO 02/46191; WO 02/46192; WO 02/46193; WO 02/46749 WO 02/102377; WO 03/020889; WO 03/043572 and WO 03/045391.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain benzimidazole derivatives (such as those described in U.S. Pat. No. 6,387,938), and certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/085905).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG and are described, for example, in International Patent Publication No. WO 00/75304.

Small molecule IRM compounds suitable for use as a TLR agonist in immunostimulatory combinations of the invention include compounds having a 2-aminopyridine fused to a five membered nitrogen-containing heterocyclic ring. Such compounds include, for example, imidazoquinoline amines including but not limited to substituted imidazoquinoline amines such as, for example, aminoalkyl-substituted imidazoquinoline amines, amide-substituted imidazoquinoline amines, sulfonamide-substituted imidazoquinoline amines, urea-substituted imidazoquinoline amines, aryl ether-substituted imidazoquinoline amines, heterocyclic ether-substituted imidazoquinoline amines, amido ether-substituted imidazoquinoline amines, sulfonamido ether-substituted imidazoquinoline amines, urea-substituted imidazoquinoline ethers, and thioether-substituted imidazoquinoline amines; tetrahydroimidazoquinoline amines including but not limited to amide-substituted tetrahydroimidazoquinoline amines, sulfonamide-substituted tetrahydroimidazoquinoline amines, urea-substituted tetrahydroimidazoquinoline amines, aryl ether-substituted tetrahydroimidazoquinoline amines, heterocyclic ether-substituted tetrahydroimidazoquinoline amines, amido ether-substituted tetrahydroimidazoquinoline amines, sulfonamido ether-substituted tetrahydroimidazoquinoline amines, urea-substituted tetrahydroimidazoquinoline ethers, and thioether-substituted tetrahydroimidazoquinoline amines; imidazopyridine amines including but not limited to amide-substituted imidazopyridine amines, sulfonamido-substituted imidazopyridine amines, urea-substituted imidazopyridine amines; aryl ether-substituted imidazopyridine amines, heterocyclic ether-substituted imidazopyridine amines, amido ether-substituted imidazopyridine amines, sulfonamido ether-substituted imidazopyridine amines, urea-substituted imidazopyridine ethers, and thioether-substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; and thiazolonaphthyridine amines.

In certain embodiments, the TLR agonist may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the TLR agonist can be a sulfonamide-substituted imidazoquinoline amine. In alternative embodiments, the TLR agonist can be a urea-substituted imidazoquinoline ether. In another alternative embodiment, the TLR agonist can be an aminoalkyl-substituted imidazoquinoline amine.

In one particular embodiment, the TLR agonist is 4-amino-.alpha.,.alpha.,2-trimethyl-1H-imidazo[4,5-c]quinolin-1-ethanol. In an alternative particular embodiment, the TLR agonist is N-(2-{2-[4-amino-2-(2-methoxyethyl)-1H-imidazo[4,5-c]quinolin-1-yl]ethoxy-}ethyl)-N-methylmorpholine-4-carboxamide. In another alternative embodiment, the TLR agonist is 1-(2-amino-2-methylpropyl)-2-(ethoxymethyl-)-1H-imidazo[4,5-c]quinolin-4-amine. In another alternative embodiment, the TLR agonist is N-[4-(4-amino-2-ethyl-1H-imidazo[4,5-c]quinolin-1-yl)b-utyl]methanesulfonamide. In yet another alternative embodiment, the TLR agonist is N-[4-(4-amino-2-propyl-1H-imidazo[4,5-c]quinolin-1-yl)butyl]me-thanesulfonamide.

In certain alternative embodiments, the TLR agonist may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an aminoalkyl-substituted imidazoquinoline amine, an amide-substituted imidazoquinoline amine, a sulfonamide-substituted imidazoquinoline amine, a urea-substituted imidazoquinoline amine, an aryl ether-substituted imidazoquinoline amine, a heterocyclic ether-substituted imidazoquinoline amine, an amido ether-substituted imidazoquinoline amine, a sulfonamido ether-substituted imidazoquinoline amine, a urea-substituted imidazoquinoline ether, or a thioether-substituted imidazoquinoline amines. As used herein, substituted imidazoquinoline amines specifically and expressly exclude 1-(2-methylpropyl)-1H-imidazo[4,5-c]quinolin-4-amin-e and 4-amino-.alpha.,.alpha.-dimethyl-2-ethoxymethyl-1H-imidazo[4,5-c]qui-nolin-1-ethanol.

“Therapeutically effective dosage of TNF-R agonist that elicits liver toxicity as a monotherapy” refers to dosages of a TNF-R agonist which are reported to elicit therapeutic benefits on immunity but which in clinical studies have been observed to elicit liver toxicity at least in some subjects (in the absence of co-administration of type 1 interferon and/or TLR agonist).

“TNF/R” or “TNF/TNF-R” generally refers to any member of either the Tumor Necrosis Factor (TNF) Superfamily or the Tumor Necrosis Factor Receptor (TNFR) Superfamily. The TNF Superfamily includes, for example, CD40 ligand, OX40 ligand, 4-1BB ligand, CD27, CD30 ligand (CD153), TNF-.alpha., TNF-.beta., RANK ligand, LT-.alpha., LT-.beta., GITR ligand, and LIGHT. The TNFR Superfamily includes, for example, CD40, OX40, 4-1BB, CD70 (CD27 ligand), CD30, TNFR2, RANK, LT-.beta.R, HVEM, GITR, TROY, and RELT. “TNF/R agonist” refers to a compound that acts as an agonist of a member of either the TNF Superfamily or the TNFR Superfamily. Unless otherwise indicated, reference to a TNF/R agonist compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers. Also, a compound may be identified as an agonist of a particular member of either superfamily (e.g., a CD40 agonist).

“TNF-R Agonist” or TNF/TNF-R Agonist” herein includes any suitable agonist of any member of either the TNF Superfamily or the TNF-R Superfamily that elicits toxicity, e.g., liver toxicity that is prevented or alleviated by administering such agonist in conjunction with at least one TLR agonist and/or type 1 interferon. In many cases, a member of one Superfamily can be an agonist of a complementary member of the other Superfamily. For example, CD40 ligand (a member of the TNF Superfamily) can act as an agonist of CD40 (a member of the TNFR Superfamily), and CD40 can act as an agonist of CD40 ligand. Thus, suitable TNF/R agonists include, for example, CD40 ligand, OX40 ligand, 4-1BB ligand, CD27, CD30 ligand (CD153), TNF-.alpha., TNF-.beta., RANK ligand, LT-.alpha., LT-.beta., GITR ligand, LIGHT, CD40, OX40, 4-1BB, CD70 (CD27 ligand), CD30, TNFR2, RANK, LT-.beta.R, HVEM, GITR, TROY, and RELT. Additionally, suitable TNF/R agonists include certain agonistic antibodies raised against a TNF/R (e.g., IC10 and FGK4.5, each of which was raised against mouse CD40).

“TNF-R agonist monotherapy” herein refers to a therapeutic regimen involving the administration of at least one TNF-R agonist, e.g., a CD40 agonist that does not include the concomitant administration of a TLR agonist and/or type 1 interferon. Typically such monotherapy may elicit liver toxicity in some subjects.

“Treatment site” refers to the site of a particular treatment. Depending upon the particular treatment, the treatment site may be an entire organism (e.g., a systemic treatment) or any portion of an organism (e.g., a localized treatment).

“Type I interferon” refers, collectively, to IFN-.alpha., IFN-.beta., IFN-omega, et al. or any mixture or combination thereof. In the present invention the term “type 1 interferon” encompasses any type 1 interferon which elicits an enhanced CD8+ immune response when administered proximate to or in combination with a TNF-R agonist, preferably a CD40 agonist. This includes alpha interferons, beta interferons and other types of interferons classified as type 1 interferons. Particularly, this includes epsilon interferon, zeta interferon, and tau interferons such as tau 1, 2, 3, 4, 5, 6, 7, 8, 9, and 10. Also, this includes variants thereof such as fragments, consensus interferons which mimic the structure of different type 1 interferon molecules such as alpha interferons, PEGylated versions thereof, type 1 interferons with altered glycosylation because of recombinant expression or mutagenesis, and the like. Those skilled in the art are well aware of different type I interferons including those that are commercially available and in use as therapeutics. Preferably the type 1 interferon will comprise a human type 1 interferon and most preferably a human alpha interferon.

“Vaccine” refers to a pharmaceutical composition that includes an antigen. A vaccine may include components in addition to the antigen such as, for example, one or more adjuvants, a carrier, etc. In some embodiments the TLR agonist will comprise a whole virus or microorganism which may be engineered to express a desired antigen. In some embodiments the microorganism or virus which functions as a TLR agonist may be genetically engineered to express a CD40 agonist or 4-1BB agonist and/or a desired antigen thereby providing the CD40 or 4-1BB agonist, TLR agonist and optional antigen in a single microbial or viral vehicle thereby facilitating administration to a host having a condition wherein enhanced antigen specific cellular immune response are desirably elicited.

Therefore, the invention provides improved (safer and more efficacious) therapies including tumor and infectious disease vaccines involving the administration of a TNF-R agonist and optionally an antigen, whereby the improvement (reduced or eliminated liver toxicity) is attained by the co-administration of the TNF-R agonist with an amount of at least one TLR agonist and/or type 1 interferon sufficient to eliminate or reduce adverse toxicity that may otherwise result if the same dosage of the TNF-R agonist, e.g., a CD40 agonist is utilized as a monotherapy. When the inventors herein state that a dosage of TNF-R agonist is toxic at a particular dosage, it is intended to mean that this dosage has been observed in clinical trials to elicit liver toxicity e.g., as manifested by an increase in some liver enzymes (transaminases) when used as a monotherapy (without TLR and/or type 1 interferon). Methods for measuring liver toxicity of drugs during clinical trials are well known as this is a side effect of many potential therapeutics which if significant enough may contravene the therapeutic use of a compound.

These therapies will include in particular conditions in which eliciting an antigen specific immune response is desirably elicited, for example a person with a chronic disease such as cancer or an infectious or allergic disorder producing said composition.

Still further the invention provides therapeutic compositions comprising an amount of said TNF-R agonist that has been found to elicit liver toxicity in some subjects (if used as a monotherapy), an amount of at least one type 1 interferon and/or TLR agonist sufficient to prevent or alleviate said liver toxicity, and optionally an antigen (or a nucleic acid sequence(s) that provides for the expression thereof in a suitable host, preferably human), suitable for the treatment of a disease, e.g., a diseases wherein eliciting an enhanced antigen-specific cellular immune response is therapeutically warranted.

Particularly, the invention provides improved (safer and more efficacious) methods of immunotherapy comprising the administration of the subject agonist and/or cytokine combination to a host in need of such treatment in order to elicit an enhanced antigen specific cellular immune response. In preferred embodiments these compositions or polypeptide conjugates or nucleic acid sequences encoding these agonists and cytokine combinations will be administered to a subject with or at risk of developing a cancer, an infection, particularly a chronic infectious diseases e.g., involving a virus, bacteria or parasite; or an autoimmune, inflammatory or allergic condition. For example, the invention may be used to elicit antigen specific cellular immune responses against HIV, lung cancer or melanoma. HIV is a well recognized example of a disease wherein protective immunity almost certainly will require the generation of potent and long-lived cellular immune responses against the virus. In addition lung cancer and melanoma are both virulent cancers that result in thousands of deaths annually and for which improved and safe therapies are desired.

Thus, this invention provides for the development of potent yet safe therapeutic therapeutics, e.g., vaccines against HIV and compositions for treating other chronic infectious diseases involving viruses, bacteria, fungi or parasites as well as proliferative diseases such as cancer, autoimmune diseases, allergic disorders, and inflammatory diseases.

### APPLICATIONS OF THE INVENTION

The present invention provides improved methods of therapy involving the administration of at least one TNF-R agonist, e.g., a CD40 agonist such as a CD40 agonistic antibody or a soluble CD40L polypeptide, fragment or conjugate containing, whereby the toxicity (liver toxicity) associated with such agonist if used as a monotherapy at a desired therapeutic dosage is reduced or eliminated by the further administration of a effective amount of at least one TLR agonist and/or type 1 interferon. (In this context “effective” means that the type 1 interferon or TLR agonist eliminates or reduces liver toxicity of the TNF-R agonist.) The TLR agonist and/or type 1 interferon and TNF/R agonist are provided (or administered, as appropriate to the form of the immunostimulatory conjugate containing or encoding these moieties) in an amount effective to increase the immune response to a particular antigen. Also, as mentioned, the amount of the TNF-R agonist, e.g., CD40 agonist will typically comprise a dosage that elicits toxicity (liver toxicity) in at least some subjects if administered as a monotherapy. In addition, the amount of the TLR agonist and/or type 1 interferon will be an amount sufficient to prevent or alleviate said toxicity and will be administered before, during or after TNF-R agonist administration.

For example, the TLR agonist can be administered in an amount from about 100 ng/kg to about 100 mg/kg. In many embodiments, the TLR agonist is administered in an amount from about 10 .mu·g/kg to about 10 mg/kg. In some embodiments, the TLR agonist is administered in an amount from about 1 mg/kg to about 5 mg/kg. The particular amount of TLR agonist that constitutes an amount effective to increase the immune response to a particular antigen, however, depends to some extent upon certain factors including but not limited to the particular TLR agonist being administered; the particular antigen being administered and the amount thereof; the particular TNF/R agonist being administered and the amount thereof; the state of the immune system (e.g., suppressed, compromised, stimulated); the method and order of administration of the TLR agonist, the TNF/R agonist, and the antigen; the species to which the formulation is being administered; and the desired therapeutic result. Accordingly it is not practical to set forth generally the amount that constitutes an effective amount of the TLR agonist. Those of ordinary skill in the art, however, can readily determine the appropriate amount with due consideration of such factors.

The amount of the type 1 interferon will be one sufficient to prevent or alleviate the toxicity of the TNF-R agonist if administered as a monotherapy. As shown herein the toxicity of e.g., CD40 agonists can be alleviated if the CD40 agonist is administered in conjunction with a type 1 interferon or a TLR agonist. Thereby, the invention provides for more effective CD40 agonist therapies as the CD40 agonist can be administered at higher dosages than heretofore described. For example the MTD (maximum tolerated dosage) of CD40L polypeptide if co-administered with a type 1 interferon or a TLR agonist may exceed 0.1 mg/kg/day by at least 1.5 fold, more preferably by at least 2-5 fold, or even 10-fold or more thereby permitting the CD40L polypeptide to be administered at MTD amounts ranging from at least about 0.15 mg/kg/day to 1.0 mg/kg/day or higher. This will result in more effective CD40L therapies such as in the treatment of CD40 associated malignancies and other treatments disclosed herein. In addition the present invention will reduce toxicity of CD40 agonist antibody therapies and facilitate the administration of CD40 agonist antibody dosages higher than heretofore suggested. Particularly, as noted above it has been reported that the MTD for an agonistic CD40L antibody reported by Vonderheide et al., J. Clin. Immunol. 25(7):876-883 (2007) was 0.3 mg/kg and that dosages in excess resulted in transient liver toxicity, venous thromboembolism, grade 3 headaches and cytokine release and associated toxicity and adverse side effects such a fever and chills. Co-administration of the CD40 agonist antibody in association with type 1 interferon or a TLR agonist potentially allows for the MTD antibody amount to be substantially increased, e.g. by 1.5-15 or even 5-10 fold without adverse effects. Thereby the MTD amount for the CD40 agonistic antibody may be increased to about 0.45 mg/kg to about 3.0 mg/kg or even higher. Thus the invention includes the co-administration of a CD40 agonist with an amount of type 1 interferon or TLR agonist sufficient to reduce toxic effects such as liver toxicity that would otherwise potentially result at the particular CD40 agonist dosage amount.

With respect to the type 1 interferon, the amount may vary from about 1.×10.sup.3 units of activity (U) to about 1.×10 U, more typically from about 10.sup.4 U to about 10.sup.8 U.

The amount of the agonistic antibody or CD40L polypeptide may vary from about 0.00001 grams to about 5 grams, more typically from about 0.001 grams to about 1 gram. As noted above, a preferred MTD will exceed 0.3 mg/kg and may range from about 0.45 mg/kg to about 3 mg/kg. If the therapeutic method involves the administration of an antigen this may be administered at amounts ranging from about 0.0001 grams to about 50 grams, more typically from about 0.1 grams to about 10 grams. As noted, these moieties may be administered in the same or different formulations. If administered separately the moieties may be administered in any order, typically within several hours of each other, more typically substantially proximate in time.

The TNF/R agonist, e.g. a CD40 agonist may be administered in an amount from about 100 ng/kg to about 100 mg/kg. In certain embodiments, the TNF/R agonist is administered in an amount from about 10 .mu·g/kg to about 10 mg/kg. In some embodiments, the TNF/R agonist is administered in an amount from about 1 mg/kg to about 5 mg/kg. The particular amount of TNF/R agonist that constitutes an amount effective to increase the immune response to a particular antigen, however, depends to some extent upon certain factors including but not limited to the particular TNF/R agonist being administered; the particular TLR agonist being administered and the amount thereof; the particular antigen being administered and the amount thereof; the state of the immune system; the method and order of administration of the TLR agonist, the TNF/R agonist, and the antigen; the species to which the formulation is being administered; and the desired therapeutic result. Accordingly it is not practical to set forth generally the amount that constitutes an effective amount of the TNF/R agonist. Those of ordinary skill in the art, however, can readily determine the appropriate amount with due consideration of such factors.

In some embodiments, the immunostimulatory combination may further include an antigen. When present in the immunostimulatory combination, the antigen may be administered in an amount that, in combination with the other components of the combination, is effective to generate an immune response against the antigen. For example, the antigen can be administered in an amount from about 100 ng/kg to about 100 mg/kg. In many embodiments, the antigen may be administered in an amount from about 10 .mu·g/kg to about 10 mg/kg. In some embodiments, the antigen may be administered in an amount from about 1 mg/kg to about 5 mg/kg. The particular amount of antigen that constitutes an amount effective to generate an immune response, however, depends to some extent upon certain factors such as, for example, the particular antigen being administered; the particular TLR agonist being administered and the amount thereof; the particular TNF/R agonist being administered and the amount thereof; the state of the immune system; the method and order of administration of the TLR agonist, the TNF/R agonist, and the antigen; the species to which the formulation is being administered; and the desired therapeutic result. Accordingly, it is not practical to set forth generally the amount that constitutes an effective amount of the antigen. Those of ordinary skill in the art, however, can readily determine the appropriate amount with due consideration of such factors.

When present, the antigen may be administered simultaneously or sequentially with any component of the immunostimulatory combination. Thus, the antigen may be administered alone or in a mixture with one or more adjuvants (including, e.g., a TLR agonist, a type 1 interferon and/or TNF/R agonist). In some embodiments, an antigen may be administered simultaneously (e.g., in a mixture) with respect to one adjuvant, but sequentially with respect to one or more additional adjuvants.

Sequential co-administration of an antigen and other components of an immunostimulatory combination can include cases in which the antigen and at least one other component of the immunostimulatory combination are administered so that each is present at the treatment site at the same time, even though the antigen and the other component are not administered simultaneously. Sequential co-administration of the antigen and the other components of the immunostimulatory combination also can include cases in which the antigen or at least one of the other components of the immunostimulatory combination is cleared from a treatment site, but at least one cellular effect of the cleared antigen or other component (e.g., cytokine production, activation of a certain cell population, etc.) persists at the treatment site at least until one or more additional components of the combination are administered to the treatment site. Thus, it may be possible that an immunostimulatory combination of the invention can, in certain circumstances, include one or more components that never exist in a mixture with another component of the combination.

The antigen can be any material capable of raising a TH1 immune response, which may include one or more of, for example, a CD8+ T cell response, an NK T cell response, a .gamma./.delta. T cell response, or a TH1 antibody response. Suitable antigens include but are not limited to peptides; polypeptides; lipids; glycolipids; polysaccharides; carbohydrates; polynucleotides; prions; live or inactivated bacteria, viruses or fungi; and bacterial, viral, fungal, protozoal, tumor-derived, or organism-derived antigens, toxins or toxoids.

Furthermore, it is contemplated that certain currently experimental antigens, especially materials such as recombinant proteins, glycoproteins, and peptides that do not raise a strong immune response, can be used in connection with adjuvant combinations of the invention. Exemplary experimental subunit antigens include those related to viral disease such as adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever.

In certain embodiments, the antigen may be a cancer antigen or a tumor antigen. The terms cancer antigen and tumor antigen are used interchangeably and refer to an antigen that is differentially expressed by cancer cells. Therefore, cancer antigens can be exploited to differentially target an immune response against cancer cells. Cancer antigens may thus potentially stimulate tumor-specific immune responses. Certain cancer antigens are encoded, though not necessarily expressed, by normal cells. Some of these antigens may be characterized as normally silent (i.e., not expressed) in normal cells, those that are expressed only at certain stages of differentiation, and those that are temporally expressed (e.g., embryonic and fetal antigens). Other cancer antigens can be encoded by mutant cellular genes such as, for example, oncogenes (e.g., activated ras oncogene), suppressor genes (e.g., mutant p53), or fusion proteins resulting from internal deletions or chromosomal translocations. Still other cancer antigens can be encoded by viral genes such as those carried by RNA and DNA tumor viruses.

Cancers or tumors and specific tumor antigens associated with such tumors (but not exclusively), include acute lymphoblastic leukemia (etv6, aml1, cyclophilin b), B cell lymphoma (Ig-idiotype), glioma (E-cadherin, .alpha.-catenin, .beta.-catenin, .gamma.-catenin, p120ctn), bladder cancer (p21ras), biliary cancer (p21 ras), breast cancer (MUC family, HER2/neu, c-erbB-2), cervical carcinoma (p53, p21ras), colon carcinoma (p21ras, HER2/neu, c-erbB-2, MUC family), colorectal cancer (Colorectal associated antigen (CRC)-CO17-1A/GA733, APC), choriocarcinoma (CEA), epithelial cell cancer (cyclophilin b), gastric cancer (HER2/neu, c-erbB-2, ga733 glycoprotein), hepatocellular cancer (.alpha.-fetoprotein), Hodgkins lymphoma (Imp-1, EBNA-1), lung cancer (CEA, MAGF-3, NY-ESO-1), lymphoid cell-derived leukemia (cyclophilin b), melanoma (p5 protein, gp75, oncofetal antigen, GM2 and GD2 gangliosides, Melan-A/MART-1, cdc27, MAGE-3, p21ras, gp100.sup.Pmel117), myeloma (MUC family, p21.ras), non-small cell lung carcinoma (HER2/neu, c-erbB-2), nasopharyngcal cancer (Imp-1, EBNA-1), ovarian cancer (MUC family, HER2/neu, c-erbB-2), prostate cancer (Prostate Specific Antigen (PSA) and its antigenic epitopes PSA-1, PSA-2, and PSA-3, PSMA, HER2/neu, c-erbB-2, ga733 glycoprotein), renal cancer (HER2/neu, c-erbB-2), squamous cell cancers of the cervix and esophagus (viral products such as human papilloma virus proteins), testicular cancer (NY-ESO-1), and T cell leukemia (HTLV-1 epitopes).

Immunostimulatory combinations of the invention that include an antigen may form a vaccine. Such vaccines can contain additional pharmaceutically acceptable ingredients, excipients, carriers, and the like well known to those skilled in the art.

Immunostimulatory combinations of the invention can be administered to animals, e.g., mammals (human and non-human), fowl, and the like according to conventional methods well known to those skilled in the art (e.g., orally, subcutaneously, nasally, topically).

The invention also provides therapeutic and/or prophylactic methods that include administering an immunostimulatory combination of the invention to a subject.

Unless a specific sequence of administration is provided, components of the immunostimulatory combination may be administered simultaneously with the antigen (together in admixture or separately, e.g., orally or by separate injection) or subsequent to administering one or more other components of the immunostimulatory combination. For example, a TLR agonist or a type 1 interferon and a TNF/R agonist may be administered simultaneously with one another or sequentially with respect to each other. Also, when an antigen is present as a component of the immunostimulatory combination, it may be administered simultaneously with, or sequentially with respect to, any other component of the combination.

Components of the immunostimulatory combination can be administered simultaneously or sequentially in any order. When the components are administered simultaneously, they can be administered in a single formulation or in distinct formulations. When administered as distinct formulations, whether simultaneously or sequentially, the components may be administered at a single site or at separate sites. Also, when administered as distinct formulations, each formulation may be administered using a different route. Suitable routes of administration include but are not limited to transdermal or transmucosal absorption, injection (e.g., subcutaneous, intraperitoneal, intramuscular, intravenous, etc.), ingestion, inhalation, and the like. When administered sequentially, the time between administration of the components can be determined, at least in part, by certain factors such as, for example, the length of time a particular component persists, either systemically or at the administration site; or the length of time that the cellular effects of the component persist, either systemically or at the administration site, even after the component has been cleared.

Certain small molecule IRM compounds can induce biosynthesis of antiviral cytokines. Therefore, for certain embodiments that include a live viral antigen and a small molecule IRM compound as the TLR agonist component of the immunostimulatory combination, it may be desirable to administer the antigen prior to administering the IRM compound so that the viral infection can be established.

In one aspect, methods of the invention can include administering a vaccine including an immunostimulatory combination of the invention to induce a TH1 immune response in a subject. As noted above, certain small molecule IRMs, alone, may be useful as a vaccine adjuvant. An immunostimulatory combination that includes a TLR agonist (e.g., a small molecule IRM) and a TNF/R agonist can provide an even greater immune response than either an antigen alone, an antigen combined with a TLR agonist, or an antigen combined with a TNF/R agonist. In some cases, an immunostimulatory combination that includes a TLR agonist and a TNF/R agonist can synergistically increase an immune response compared to either a TLR agonist or TNF/R agonist.

Methods of the invention also include inducing an immune response from cells of the immune system regardless of whether the cells are in vivo or ex vivo. Thus, an immunostimulatory combination of the invention may be useful as a component of a therapeutic vaccine, a component of a prophylactic vaccine, or as an immunostimulatory factor used in ex vivo cell culture. When used to elicit an immune response ex vivo, the immune cells activated ex vivo may be reintroduced into a patient. Alternatively, factors secreted by the activated immune cells in the cell culture, (e.g., antibodies, cytokines, co-stimulatory factors, and the like) may be collected for investigative, prophylactic, or therapeutic uses.

Methods of the invention also include activating naive CD8+ T cells in an antigen-specific manner in vivo. The population of activated antigen-specific CD8+ T cells produced in response to co-administration of an antigen and an immunostimulatory combination-1-whether or not the antigen is explicitly a component of the immunostimulatory combination—may be divided into two functionally distinct sub-populations. One population of antigen-specific CD8+ T cells includes effector T cells,—CD8+ T cells actively engaged in providing a cell-mediated immune response. A second population of antigen-specific CD8+ T cells includes memory T cells, CD8+ T cells that are not themselves involved in providing an immune response, but can be readily induced to become antigen-specific effector cells upon a later contact with the same antigen. Activation of CD8+ T cells according to the following method may induce expansion of antigen-specific CD8+ effector T cells, generate antigen-specific CD8+ memory T cells, or both.

An immunostimulatory combination that includes an antigen may be administered to a subject. After sufficient incubation in the subject, CD8.+ T cells will mature to antigen-specific CD8+ effector T cells in response to the immunization. A greater percentage of CD8+ effector T cells will be antigen-specific in subjects immunized with an immunostimulatory combination that includes a TLR agonist and a TNF/R agonist compared to subjects immunized with only antigen, antigen and a TNF/R agonist, or antigen and a TLR agonist. Generally, the incubation time between immunization and the generation of CD8+ effector T cells is from about 4 days to about 12 days. In certain embodiments, CD8+ effector T cells may be generated in about 5 days after immunization. In other embodiments, CD8.+ effector T cells may be generated in about 7 days after immunization.

If the antigen is a protein, it may not be necessary to administer the entire protein to the subject. Thus, a method that includes administering to a subject an immunostimulatory combination of the invention may be used to elicit an antigen-specific response in CD8+ cytotoxic T lymphocytes (CTLs) of the subject. Such a response may be directed against many conditions including, for example, tumors and virus-infected cell populations. In some embodiments of the invention, a vaccine of the invention may be administered prophylactically to provide a subject with a protective antigen-specific cell-mediated immunity directed against, for example, tumors and/or viral infections.

In an alternative embodiment, immunostimulatory combinations of the present invention may be used to develop antigen-specific CD8.+ memory T cells in vivo. The antigen-specific CD8+ memory T cells may be capable of generating a secondary TH1 immune response upon a second exposure to the antigen. CD8+ effector T cells may be generated from the re-activated CD8+ memory T cells in as little as 2 hours after re-exposure to the antigen. The second exposure to the antigen may be by immunization (i.e., a booster immunization) or natural exposure.

An immunostimulatory combination of the invention can be used to therapeutically treat a condition treatable by a cell-mediated immune response. Such a combination can contain at least a therapeutically effective amount of a TLR agonist and a therapeutically effective amount of a TNF/R agonist. In many embodiments, a therapeutic combination can further include a therapeutically effective amount of an antigen.

A therapeutic combination can be provided in further combination with one or more pharmaceutically acceptable carriers. Because the TLR agonist and/or type 1 interferon, TNF/R agonist, and antigen (if present in the combination) may be co-administered sequentially, at different sites, and/or by different routes, a therapeutic combination may be provided in two or more formulations. When provided in two or more formulations, each formulation can include a carrier similar or different than the carrier or carriers included in the remaining formulations. Alternatively, the TLR agonist, and/or type 1 interferon TNF/R agonist, and antigen (if present in the combination) may be provided in a single formulation, which can include a single carrier or a combination of carriers.

Each component or mixture of components may be administered in any suitable conventional dosage form such as, for example, tablets, lozenges, parenteral formulations, syrups, creams, ointments, aerosol formulations, transdermal patches, transmucosal patches and the like.

Therapeutic immunostimulatory combinations can be administered as the single therapeutic agent in the treatment regimen. Alternatively, a therapeutic immunostimulatory combination of the invention may be administered in combination with another therapeutic combination of the invention, with one or more pharmaceutical compositions, or with other active agents such as antivirals, antibiotics, additional IRM compounds, etc.

Because of their ability to induce the TH1 immune response and generate a pool of CD8+ effector T cells, certain immunostimulatory combinations of the invention can be particularly useful for treating viral diseases and tumors. This immunomodulating activity suggests that immunostimulatory combinations and vaccines of the invention are useful in treating conditions such as, but not limited to:
(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);
(b) bacterial diseases such as, for example, diseases resulting from infection by bacteria of, for example, the genus Escherichia, Enterobacter, Salmonella, Staphylococcus, Shigella, Listeria, Acrobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Hacmophilus, or Bordetella;
(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and
(d) neoplastic diseases, such as, for example, intraepithelial neoplasias, cervical dysplasia, actinic keratosis, basal cell carcinoma, squamous cell carcinoma, renal cell carcinoma, Kaposi's sarcoma, lung cancer, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers (e.g., cancers identified above); and
(e) TH2-mediated, atopic, and autoimmune diseases, such as atopic dermatitis or eczema, eosinophilia, asthma, allergy, allergic rhinitis, systemic lupus erythematosus, essential thrombocythaemia, multiple sclerosis, Ommen's syndrome, discoid lupus, alopecia areata, inhibition of keloid formation and other types of scarring, and enhancing would healing, including chronic wounds.

Some embodiments of the immunostimulatory combinations of the invention also may be useful as a vaccine adjuvant for use in conjunction with any material that raises either humoral and/or cell mediated immune response, such as, for example, live viral, bacterial, or parasitic antigens; inactivated viral, tumor-derived, protozoal, organism-derived, fungal, or bacterial antigens, toxoids, toxins; self-antigens; polysaccharides; proteins; glycoproteins; peptides; cellular vaccines; DNA vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Immunostimulatory combinations of the invention may also be particularly helpful in individuals having compromised immune function. For example, IRM, compounds may be used for treating the opportunistic infections and tumors that occur after suppression of cell mediated immunity in, for example, transplant patients, cancer patients and HIV patients.

The invention also provides a method of treating a viral infection in an animal and a method of treating a neoplastic disease in an animal comprising administering a therapeutically effective amount of an immunostimulatory combination of the invention to the animal. A therapeutically effective amount to treat or inhibit a viral infection is an amount that will cause a reduction in one or more of the manifestations of viral infection, such as viral lesions, viral load, rate of virus production, and mortality as compared to untreated control animals. A therapeutically effective amount of a combination to treat a neoplastic condition is an amount that will cause, for example, a reduction in tumor size, a reduction in the number of tumor foci, or slow the growth of a tumor, as compared to untreated animals.

In one particular embodiment, an immunostimulatory combination of the invention may be used to inhibit tumor growth in vivo. Subjects having tumor cells expressing a particular antigen may be immunized with a therapeutic combination that contains a TLR agonist, a TNF/R agonist, and, optionally, the antigen. In some embodiments, the therapy can include an initial immunization and a second booster immunization. Tumors taken from subjects immunized with a therapeutic combination of the invention were generally smaller than the tumors harvested from either (a) non-immunized subjects, or (b) subjects immunized with only the antigen.

Treatments according to the present invention may include one or more than one immunization. When the treatment includes more than one immunization, the treatment can include any suitable number of immunizations administered at any suitable frequency. The number and frequency of immunizations in a treatment regimen depend at least in part upon one or more factors including but not limited to the condition being treated and the stage thereof, the state of the subject's immune system, the particular TLR agonist or type 1 interferon being administered and the amount thereof, the particular TNF/R agonist being administered and the amount thereof, and the particular antigen being administered (if present) and the amount thereof.

As mentioned, in some embodiments, therapeutic combinations of the invention may not require an antigen component. For certain conditions (e.g., B cell lymphoma or chronic bacterial or viral infections), effective treatment may be obtained using an immunostimulatory combination that does not include an antigen. Such conditions may be treatable in this way because, for example, the condition may provide a sufficient quantity or variety of condition-specific antigens to generate a cell-mediated immune response capable of treating the condition.

The TLR agonist and/or type 1 interferon and TNF/R agonist are provided (or administered, as appropriate to the form of the immunostimulatory combination) in an amount effective to increase the immune response to a particular antigen and at a dosage wherein the TNF-R agonist may elicit liver toxicity as a monotherapy.

For example, the TLR agonist can be administered in an amount from about 100 ng/kg to about 100 mg/kg. In many embodiments, the TLR agonist is administered in an amount from about 10 .mu·g/kg to about 10 mg/kg. In some embodiments, the TLR agonist is administered in an amount from about 1 mg/kg to about 5 mg/kg. The particular amount of TLR agonist that constitutes an amount effective to increase the immune response to a particular antigen, however, depends to some extent upon certain factors including but not limited to the particular TLR agonist being administered; the particular antigen being administered and the amount thereof; the particular TNF/R agonist being administered and the amount thereof; the state of the immune system (e.g., suppressed, compromised, stimulated); the method and order of administration of the TLR agonist, the TNF/R agonist, and the antigen; the species to which the formulation is being administered; and the desired therapeutic result. Accordingly it is not practical to set forth generally the amount that constitutes an effective amount of the TLR agonist. Those of ordinary skill in the art, however, can readily determine the appropriate amount with due consideration of such factors.

Also, for example, the TNF/R agonist may be administered in an amount from about 100 ng/kg to about 100 mg/kg. In certain embodiments, the TNF/R agonist is administered in an amount from about 10 .mu·g/kg to about 10 mg/kg. In some embodiments, the TNF/R agonist is administered in an amount from about 1 mg/kg to about 5 mg/kg. The particular amount of TNF/R agonist that constitutes an amount effective to increase the immune response to a particular antigen, however, depends to some extent upon certain factors including but not limited to the particular TNF/R agonist being administered; the particular TLR agonist being administered and the amount thereof; the particular antigen being administered and the amount thereof; the state of the immune system; the method and order of administration of the TLR agonist, the TNF/R agonist, and the antigen; the species to which the formulation is being administered; and the desired therapeutic result. Accordingly it is not practical to set forth generally the amount that constitutes an effective amount of the TNF/R agonist. Those of ordinary skill in the art, however, can readily determine the appropriate amount with due consideration of such factors.

By contrast, in some embodiments, the immunostimulatory combination may further include an antigen. When present in the immunostimulatory combination, the antigen may be administered in an amount that, in combination with the other components of the combination, is effective to generate an immune response against the antigen. For example, the antigen can be administered in an amount from about 100 ng/kg to about 100 mg/kg. In many embodiments, the antigen may be administered in an amount from about 10 .mu·g/kg to about 10 mg/kg. In some embodiments, the antigen may be administered in an amount from about 1 mg/kg to about 5 mg/kg.

The particular amount of antigen that constitutes an amount effective to generate an immune response, however, depends to some extent upon certain factors such as, for example, the particular antigen being administered; the particular TLR agonist being administered and the amount thereof; the particular TNF/R agonist being administered and the amount thereof; the state of the immune system; the method and order of administration of the TLR agonist, the TNF/R agonist, and the antigen; the species to which the formulation is being administered; and the desired therapeutic result. Accordingly, it is not practical to set forth generally the amount that constitutes an effective amount of the antigen. Those of ordinary skill in the art, however, can readily determine the appropriate amount with due consideration of such factors.

When present, the antigen may be administered simultaneously or sequentially with any component of the immunostimulatory combination. Thus, the antigen may be administered alone or in a mixture with one or more adjuvants (including, e.g., a TLR agonist and/or type I interferon, a TNF/R agonist, or a combination thereof). In some embodiments, an antigen may be administered simultaneously (e.g., in a mixture) with respect to one adjuvant, but sequentially with respect to one or more additional adjuvants.

Sequential co-administration of an antigen and other components of an immunostimulatory combination can include cases in which the antigen and at least one other component of the immunostimulatory combination are administered so that each is present at the treatment site at the same time, even though the antigen and the other component are not administered simultaneously. Sequential co-administration of the antigen and the other components of the immunostimulatory combination also can include cases in which the antigen or at least one of the other components of the immunostimulatory combination is cleared from a treatment site, but at least one cellular effect of the cleared antigen or other component (e.g., cytokine production, activation of a certain cell population, etc.) persists at the treatment site at least until one or more additional components of the combination are administered to the treatment site. Thus, it may be possible that an immunostimulatory combination of the invention can, in certain circumstances, include one or more components that never exist in a mixture with another component of the combination.

The invention also provides therapeutic and/or prophylactic methods that include administering an immunostimulatory combination of the invention to a subject.

In some embodiments the methods and compositions can be used to treat an individual at risk of having an infection or has an infection by including an antigen from the infectious agent. An infection refers to a disease or condition attributable to the presence in the host of a foreign organism or an agent which reproduce within the host. A subject at risk of having an infection is a subject that is predisposed to develop an infection. Such an individual can include for example a subject with a known or suspected exposure to an infectious organism or agent. A subject at risk of having an infection can also include a subject with a condition associated with impaired ability to mount an immune response to an infectious agent or organism, for example a subject with a congenital or acquired immunodeficiency, a subject undergoing radiation or chemotherapy, a subject with a burn injury, a subject with a traumatic injury, a subject undergoing surgery, or other invasive medical or dental procedure, or similarly immunocompromised individual.

Infections which may be treated or prevented with the vaccine compositions of this invention include bacterial, viral, fungal, and parasitic. Other less common types of infection also include are rickettsiae, mycoplasms, and agents causing scrapie, bovine spongiform encephalopathy (BSE), and prion diseases (for example kuru and Creutzfeldt-Jacob disease). Examples of bacteria, viruses, fungi, and parasites that infect humans are well know. An infection may be acute, subacute, chronic or latent and it may be localized or systemic. Furthermore, the infection can be predominantly intracellular or extracellular during at least one phase of the infectious organism's agent's life cycle in the host.

Bacteria infections against which the subject vaccines and methods may be used include both Gram negative and Gram positive bacteria. Examples of Gram positive bacteria include but are not limited to Pasteurella species, Staphylococci species, and Streptococci species. Examples of Gram negative bacteria include but are not limited to Escherichia coli, Pseudomonas species, and Salmonella species. Specific examples of infectious bacteria include but are not limited to Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae(Group B Streptococcus), Streptococcus (viridans group), Streptococcus faecalis, streptococcus bovis, Streptococcus (aenorobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium pcrfringens, Clostridium tetani, Entcrobacter acrogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that cause infections in humans include but are not limited to Retroviridae (for example human deficiency viruses, such as HIV-1 (also referred to as HTLV-III), HIV-II, LAC or IDLV-III/LAV or HIV-III and other isolates such as HIV-LP, Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses (for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses).

Examples of fungi include Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

Parasites include but are not limited to blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

As noted this invention further embraces the use of the subject therapeutic regimens and compositions in treating proliferative diseases such as cancers. Cancer is a condition of uncontrolled growth of cells which interferes with the normal functioning of bodily organs and systems. A subject that has a cancer is a subject having objectively measurable cancer cells present in the subjects' body. A subject at risk of developing cancer is a subject predisposed to develop a cancer, for example based on family history, genetic predisposition, subject exposed to radiation or other cancer-causing agent. Cancers which migrate from their original location and seed vital organs can eventually lead to the death of the subject through the functional deterioration of the affected organ. Hematopoietic cancers, such as leukemia, are able to out-compete the normal hematopoietic compartments in a subject thereby leading to hematopoietic failure (in the form of anemia, thrombocytopenia and neutropenia), ultimately causing death.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are often detected through the sole or combined use of magnetic resonance imaging (MRI), computed tomography (CT), scans, blood and platelet counts, liver function studies, chest -X-rays and bone scans in addition to the monitoring of specific symptoms.

The adjuvant combinations and compositions containing according to the invention can be used to treat a variety of cancers or subjects at risk of developing cancer, by the inclusion of a tumor-associated-antigen (TAA), or DNA encoding. This is an antigen expressed in a tumor cell. Examples of such cancers include breast, prostate, colon, blood cancers such as leukemia, chronic lymphocytic leukemia, and the like. The vaccination methods of the invention can be used to stimulate an immune response to treat a tumor by inhibiting or slowing the growth of the tumor or decreasing the size of the tumor. A tumor associated antigen can also be an antigen expressed predominantly by tumor cells but not exclusively.

Additional cancers include but are not limited to basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), lymphoma including Hodgkin's lymphoma and non-Hodgkin's lymphoma; melanoma; neuroblastoma; oral cavity cancer (for example lip, tongue, mouth and pharynx); ovarian cancer; pancreatic cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system; as well as other carcinomas and sarcomas.

The adjuvant combinations and compositions containing according to the invention can also be used to treat autoimmune diseases such as multiple sclerosis, rheumatoid arthritis, type 1 diabetes, psoriasis or other autoimmune disorders. Other autoimmune disease which potentially may be treated with the vaccines and immune adjuvants of the invention include Crohn's disease and other inflammatory bowel diseases such as ulcerative colitis, systemic lupus eythematosus (SLE), autoimmune encephalomyelitis, myasthenia gravis (MG), Hashimoto's thyroiditis, Goodpasture's syndrome, pemphigus, Graves disease, autoimmune hemolytic anemia, autoimmune thrombocytopenic purpura, scleroderma with anti-collagen antibodies, mixed connective tissue disease, polypyositis, pernicious anemia, idiopathic Addison's disease, autoimmune associated infertility, glomerulonephritis) for example crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin dependent diabetes mellitus), autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, and Guillain-Bare syndrome. Recently, arteriosclerosis and Alzheimer's disease have been recognized as autoimmune diseases. Thus, in this embodiment of the invention the antigen will be a self-antigen against which the host elicits an unwanted immune response that contributes to tissue destruction and the damage of normal tissues.

The adjuvant combinations and compositions containing according to the invention can also be used to treat asthma and allergic and inflammatory diseases. Asthma is a disorder of the respiratory system characterized by inflammation and narrowing of the airways and increased reactivity of the airways to inhaled agents. Asthma is frequently although not exclusively associated with atopic or allergic symptoms. Allergy is acquired hypersensitivity to a substance (allergen). Allergic conditions include eczema, allergic rhinitis, or coryza, hay fever, bronchial asthma, urticaria, and food allergies and other atopic conditions. An allergen is a substance that can induce an allergic or asthmatic response in a susceptible subject. There are numerous allergens including pollens, insect venoms, animal dander, dust, fungal spores, and drugs.

Examples of natural and plant allergens include proteins specific to the following genera: Canine, Dermatophagoides, Felis, Ambrosia, Lotium, Cryptomeria, Alternaria, Alder, Alinus, Betula, Quercus, Olea, Artemisia, Plantago, Parietaria, Blatella, Apis, Cupressus, Juniperus, Thuya, Chamaecyparis, Periplanet, Agopyron, Secale, Triticum, Dactylis, Festuca, Poa, Avena, Holcus, Anthoxanthum, Arrhenatherum, Agrostis, Phleum, Phalaris, Paspalum, Sorghum, and Bromis.

It is understood that the adjuvant combinations and compositions containing according to the invention can be combined with other therapies for treating the specific condition, e.g., infectious disease, cancer or autoimmune condition. For example in the case of cancer the inventive methods may be combined with chemotherapy or radiotherapy.

In some instances, it may be beneficial to include a moiety in the adjuvant which facilitates affinity purification. Such moieties include relatively small molecules that do not interfere with the function of the adjuvant combination. Alternatively, the tags may be removable by cleavage. Examples of such tags include poly-histidine tags, hemagglutinin tags, maltase binding protein, lectins, glutathione-S transferase, avidin and the like. Other suitable affinity tags include FLAG, green fluorescent protein (GFP), myc, and the like.

The subject adjuvant combinations can be administered with a physiologically acceptable carrier such as physiological saline. The composition may also include another carrier or excipient such as buffers, such as citrate, phosphate, acetate, and bicarbonate, amino acids, urea, alcohols, ascorbic acid, phospholipids, proteins such as serum albumin, ethylenediamine tetraacetic acid, sodium chloride or other salts, liposomes, mannitol, sorbitol, glycerol and the like. The adjuvants of the invention can be formulated in various ways, according to the corresponding route of administration. For example, liquid formulations can be made for ingestion or injection, gels or procedures can be made for ingestion, inhalation, or topical application. Methods for making such formulations are well known and can be found in for example, “Remington's Pharmaceutical Sciences,” 18th Ed., Mack Publishing Company, Easton Pa.

The invention also embraces DNA based vaccines. These DNAs which may encode a desired antigen and/or CD40 adjuvant may be administered as naked DNAs, or may be comprised in an expression vector such as a recombinant virus that functions as the TLR agonist. Furthermore, the subject nucleic acid sequences may be introduced into a cell of a graft prior to transplantation of the graft. This DNA preferably will be humanized to facilitate expression in a human subject.

The subject adjuvant combinations may further include a “marker” or “reporter”. Examples of marker or reporter molecules include beta lactamase, chloramphenicol acetyltransferase, adenosine deaminase, aminoglycoside phosphotransferase, dihydrofolate reductase, hygromycin B-phosphotransferase, thymidine kinase, lacZ, and xanthine guanine phosphoribosyltransferase et al.

The subject adjuvants may be expressed by a cell comprising a vector or vectors capable of directing the expression of an antigen or TNF-R agonist and/or type 1 interferon or TLR agonist, for example a cell transduced with the vector. For example a baculovirus vector can be used. Other vectors which may be used include T7 based vectors for use in bacteria, yeast expression vectors, mammalian expression vectors, viral expression vectors, and the like. Viral vectors include retroviral, adenoviral, adeno-associated vectors, herpes virus, simian virus 40, and bovine papilloma virus vectors. Also, bacterial and yeast expression vectors may be utilized.

One skilled in the art can readily select appropriate components for a particular expression system, including expression vector, promoters, selectable markers, and the like suitable for a desired cell or organism. The selection and use of various expression systems can be found for example in Ausubel et al., “Current Protocols in Molecular Biology, John Wiley and Sons, New York, N.Y. (1993); and Pouwels et al., Cloning Vectors: A Laboratory Manual”, 1985 Suppl. 1987). Also provided are eukaryotic cells that contain and express the subject DNA constructs.

In the case of cell transplants, the cells can be administered either by an implantation procedure or with a catheter-mediated injection procedure through the blood vessel wall. In some cases, the cells may be administered by release into the vasculature, from which the cells subsequently are distributed by the blood stream and/or migrate into the surrounding tissue.

In the case of CD40 agonists as the TNF-R agonist, such agonist will preferably comprise an agonistic anti-CD40 antibody or fragment thereof that specifically binds CD40, preferably murine or human CD40, or a CD40L protein, derivative, multimer such as a trimeric CD40L or 4-1BB ligand conjugate. As used herein, the term “antibody” is used in its broadest sense to include polyclonal and monoclonal antibodies, as well as antigen binding fragments thereof. This includes Fab, F(ab′)2, Fd and Fv fragments.

The present invention is now further described based on the examples which follow.

### EXAMPLES

### Materials and Methods

Mice and Tumor Cell Lines

Male 6- to 8-week-old C57BL/6 mice were obtained from the National Cancer Institute (Bethesda, Md.) and were maintained under pathogen-free conditions. All experiments were approved by the Institutional Animal Care and Use Committee of Dartmouth College. B16.F10 melanoma cells were a kind gift from Mary Jo Turk (Dartmouth-Hitchcock Medical Center, Lebanon, N.H.) and were maintained in complete medium (RPMI 1640 containing 10% fetal calf serum, 100 U/mL penicillin, 100 μg/mL streptomycin, 2 mM glutamine, and 50 μM 2-mercaptoethanol).

Cell Lines, Antibodies, and Reagents

Mouse monoclonal antibodies (mAbs) to CD8 (53-6.7), CD4 (GK1.5), CD44 (IM7), CD127 (A7R34), CD122 (5H4), IL-2 (JES6-5H4), IFN (XMG1.2), FoxP3 (FJK-16s), Granzyme B (16G6), and the isotype control rat IgG2a were purchased from eBioscience (San Diego, Calif.) as were both brefeldin A and monensin. Anti-CD107a (1D4B) was purchased from BD Pharmingen (San Jose, Calif.). Anti-TNF (MP6-XT22) was purchased from Invitrogen (Carlsbad, Calif.). Recombinant human IL-2 was purchased from Peprotech (Rocky Hill, N.J.). Anti-CD40 (FGK45) was purchased from BioExpress (Lebanon, N.H.). Endotoxin content was less than 1 EU/mg as assessed by a quantitative chromogenic limulus amebocyte lysate kit (QCL 1000; Cambrex, East Rutherford, N.J.). The TLR7 agonist S-27609 was a gift from 3M Pharmaceuticals (St Paul, Minn.) and has been previously described.⁸ Anti-CD4 (GK1.5), anti-CD8 (2.43), and anti-NK1.1 (PK136) were produced by hybridomas, and bioreactor supernatants were purified using standard methodologies. The H2K^{b}-restricted class I peptides Ova₍₂₅₇₋₂₆₄₎ (SIINFEKL) and TRP2₍₁₈₀₋₁₈₈₎ (SVYDFFVWL) and the modified TRP2 epitope V (SIYDFFVWL) were purchased from Pepceuticals (Nottingham, United Kingdom) and were more than 90% pure. Peptides were dissolved at 5 mg/mL in DMSO and subsequently diluted in phosphate-buffered saline (PBS) for immunization.

Cell Preparation

At various times after vaccination, tissues were removed for analysis. Spleens were homogenized into single cell suspensions and peripheral blood was collected into heparinized tubes via either retro-orbital bleeds or cardiac puncture. Red blood cells were lysed with ACK Lysing Buffer (BioSource, Rockville, Md.). To isolate lymphocytes from metastatic target organs, lungs were removed and injected with RPMI containing 417.5 μg/mL Liberase CI (Roche, Indianapolis, Ind.) and 200 μg/mL DNase I (Roche), minced, and incubated at 37° C. for 30 minutes before being passed through cell strainers. Cells were washed and resuspended in 80% Percoll, overlaid with 40% Percoll, and centrifuged for 25 minutes at 400 g. Cells residing at the 80%/40% interface were collected, washed, and counted by Guava (Guava Teclmologies, Hayward, Calif.).

Tumor Challenge and Vaccinations

Mice were injected with 10⁵ B16.F10 melanoma tumor cells intravenously to establish lung metastases. Four days later, naive or tumor-bearing mice were intravenously vaccinated with 100 μg V peptide, 100 μg anti-CD40, and 100 μg of the TLR7 agonist S-27609 in various combinations as indicated. Lungs were harvested approximately 20 days later, and metastases were enumerated with the aid of a dissection microscope. Alternatively, mice were monitored for survival over the next 90 days.

In Vivo Depletion of Cell Subsets

Depletion of lymphocyte subsets was accomplished by intraperitoneal administration of 250 μg anti-CD4 (GK1.5), anti-CD8 (2.43), and anti-NK1.1 (PK136). Antibodies were delivered 4 days before the start of experimentation and weekly thereafter. Depletion was confirmed by flow cytometry and resulted in greater than 95% reduction of relevant cell types.

Flow Cytometry

Single cell suspensions were incubated with antibodies labeled with FITC, PE, PerCP, PC5, or APC. Antibodies, as listed under “Cell lines, antibodies, and reagents,” were from cBioscicnce, BD Phanningen, and Invitrogen. Four-color analyses were performed on a modified Becton Dickinson FACSCAN running CellQuest software (BD Bioscience).

Intracellular Cytokine Staining and Degranulation Assays

Cells from lung, spleen, or peripheral blood (peripheral blood lymphocytes [PBLs]) were incubated with 1 μg/mL Ova₍₂₅₇₋₂₆₄₎ or TRP2₍₁₈₀₋₁₈₈₎ peptide plus 10 U/mL IL-2 and 3 μg/mL brefeldin A in complete medium at 37° C. for 5 to 18 hours. Cells were stained with either PerCp or PC5-labeled anti-CD8 and FITC-labeled anti-CD44 antibodies prior to being fixed and rendered permeable followed by staining with either PE- or APC-labeled anti-IFN (XMG1.2), PE-labeled anti-TNF (MP6-XT22), PE-labeled anti-IL-2 (JES6-5H4), FITC-labelcd anti-CD127 (A7R34), or PE-labeled anti-granzyme B (16G6). The percentage of IFN⁺ cells was calculated by subtracting the background observed with the irrelevant peptide control. For the degranulation assay, cells were treated as above but with the inclusion of monensin and 2.5 μg/mL FITC-labeled anti-CD107a (1D4B) during the initial 5- to 18-hour incubation period.

In vivo Cytotoxicity Assay

In vivo cytolytic activity was performed as previously described. (8) Briefly, naive syngeneic splenocytes were differentially labeled with either 0.5 μM or 5 μM carboxyfluorescein succinimidyl ester (CFSE; Molecular Probes, Eugene, Oreg.) for 10 minutes at 37° C., washed, and then pulsed for 1 hour with 20 μg/mL irrelevant Ova₍₂₅₇₋₂₆₄₎ (SIINFEKL) or antigen-specific TRP2₍₁₈₀₋₁₈₈₎ peptide, respectively. Labeled and pulsed cells were subsequently mixed at a 1:1 ratio and approximately 10⁷ cells were injected intravenously. One day later, mice were killed and splenocytes were analyzed by flow cytometry. Specific lysis was calculated by first determining the ratio of the number of SIINFEKL-labeled targets to the number of TRP2-labeled targets for each mouse and percentage antigen-specific lysis was subsequently calculated as follows: % specific lysis=(1−[ratio of CFSE^{lo}/CFSE^{hi} in naive mice:ratio of CFSE^{lo}/CFSE^{hi} in immunized mice])×100.

Serum Transaminase and Histologic Analysis

Hepatocellular injury was biochemically assessed by measuring serum liver enzyme activity. Specifically, mice received 100 μg anti-CD40, 100 μg S-27609, or both intravenously or PBS as a control. Serum was harvested 24 to 72 hours later and levels of alanine aminotransferase (ALT) and aspartate aminotransferase (AST) were determined by standard clinical assays at the National Jewish Medical Center Core Lab (Denver, Colo.). For histologic analysis, livers from mice treated as above were fixed in buffered formalin, embedded in paraffin, sectioned, and stained with hematoxylin and eosin (H&E) prior to being coded and scored on a 0 to 4 scale in a blinded fashion. Numeric scores were assigned as follows: liver: 0 indicates normal liver, no lesions or hepatocellular damage noted; 1, rare portal and parenchymal infiltrates but no necrosis; 2, moderate parenchymal or portal infiltrates but no necrosis; 3, frequent and/or large portal or parenchymal infiltrates with occasional isolated islands of coagulative necrosis; and 4, extensive areas of inflammation with bridging coagulative necrosis. H&E images were acquired via an Olympus BX41 microscope (Center Valley, Pa.) using a 20×/0.05 non-oil obj ective and 100× ocular attached to an Olympus DP11 digital camera and were edited with XnView for Windows, version 1.82.2 (Reims, France).

### Statistical Analysis

Data were expressed as the mean plus or minus SEM and differences between groups were analyzed by one-tailed ANOVA and Tukey analysis unless indicated otherwise. In the case of tumor survival experiments, statistical relevance was determined using log-rank comparison. The extent of hepatitis was scored on an arbitrary scale and the resulting nonparamctric data were analyzed using the Mann-Whitney test. Probability (P) values less than 0.05 were considered statistically significant.

### Example 1

High Frequencies of Tumor-Specific, Effector CD8⁺ T Cells are Elicited Using CD40/TLR7 Agonists and Tumor-Specific Peptide

The inventors previously demonstrated that coadministration of CD40 and TLR agonists synergistically enhances expansion of antigen-specific CD8⁺ T cells to foreign antigen. (8) Herein we further show that similarly high frequencies of CD8⁺ T cells can be induced to self-antigens. Recently, a modified peptide variant of the H2K^{b}-restricted melanoma rejection self-antigen TRP2₍₁₈₀₋₁₈₈₎, termed V (SIYDFFVWL), was shown to elicit high-affinity TRP2-specific CD8⁺ T cells. (17) We reasoned that immunization with V plus agonistic CD40 antibody (CD40) and a TLR7 agonist (TLR7*) would magnify the ensuing CD8⁺ response and engender increased effector cell function. As seen in FIG. 1B, CD40 increased the relative number of CD8⁺ T cells in the peripheral blood of immunized mice, regardless of the addition of antigen, TLR7 agonist, or both (P.001 for V/CD40, V/CD40/TLR7*, and CD40/TLR7* compared with V alone). While CD40 increased polyclonal CD8⁺ responses, it failed to generate a substantial population of TRP2-specific CD8⁺ T cells (FIG. 1A,C). Only the combination of tumor antigen, CD40, and TLR7 agonist resulted in the synergistic expansion of TRP2-specific T cells. To measure cytolytic potential, we assessed the ability of these cells to degranulate, which can be measured by retention of CD107a (lysosomal-associated membrane protein-1) on the cell surface. (18) Cell-surface expression of CD107a is directly correlated with cytolytic activity. (19,20) Only approximately 4% and approximately 2% of CD8⁺ cells in either the CD40 or TLR7 agonist alone groups, respectively, expressed CD107a. However, greater than 30% of CD8⁺ cells primed with both CD40 and TLR7 agonists expressed cytolytic activity by this measure (P.001 compared with V alone). Combination treatment also led to increased lysis of peptide-pulsed targets in an in vivo cytotoxicity assay (data not shown). Together, these data demonstrate that the combination of CD40 and TLR7 agonists induces high frequencies and high total numbers of self-reactive CD8⁺ T cells with cytolytic function.

### Example 2

### Concomitant Signaling Through CD40 and TLR7 Drives Expansion of Self-Antigen-Specific CD8⁺ T Cells with Enhanced Cytolytic Activity

C57BL/6 mice were immunized intravenously with 100 μg of the tumor-associated antigen V, 100 μg CD40 FGK45, and 100 μg S-27609 in combinations as indicated. Seven days later, mice were bled and cells were restimulated in vitro with TRP2₍₁₈₀₋₁₈₈₎ to assess the ability to produce IFN and translocate CD107a as described in “Methods.” Lymphocytes were identified by forward and side scatter and subsequently gated on all CD8⁺ events. (A) Representative dot plots from vaccinated mice. The numbers in the upper right corners indicate the frequency of CD8⁺ T cells that are positive for IFN and CD44 (top row) or IFN and CD107a (bottom row). (B) Percentage of peripheral blood lymphocytes expressing the CD8 antigen. P.001 by one-tailed ANOVA (C) Quantification of the percentages of CD8⁺ cells that degranulated in response to peptide restimulation. In all cases, data presented are representative of at least 3 independent experiments. Data are plotted as means plus or minus SEM (n=8 in each group). P.001 by one-tailed ANOVA.

### Example 3

### CD40/TLR7* Vaccination Elicits Potent CD8⁺-Cell Memory

We hypothesized that coadministration of CD40 and TLR agonists would abrogate the deleterious effects of agonistic CD40-based monotherapies to engender long-term memory. To determine whether concomitant delivery of CD40 and TLR7 agonists in conjunction with tumor antigen elicits the generation of CD8⁺ T-cell memory, we vaccinated mice and analyzed effector functions 60+ days later. Vaccination with V and CD40 primed a minimal, persisting CD8⁺ effector population in the lung with limited cytolytic potential (FIG. 2A,B,D). TLR7 monotherapy failed to induce a significant pool of persisting antigen-specific CD8⁺ T cells. In contrast, vaccination with tumor antigen, CD40, and TLR7 agonist primed effector cells populating both spleen and lung (FIG. 2A,C,D). More importantly, unlike CD40 or TLR7* monotherapy, mice vaccinated with this regimen efficiently lysed peptide-pulsed targets when subjected to an in vivo cytotoxicity assay (FIG. 2B,E; P.001, compared with either V or V/CD40). In addition, the mean fluorescence intensity of IFN staining increased over that seen from CD40 treatment alone (spleen: 185±30 vs 310±22, P=0.0041; lung: 152±6 vs 253±25, P=0.0028), demonstrating that CD8⁺ T cells primed by CD40/TLR7* are more efficient in producing effector cytokines. Finally, only CD40/TLR7* plus tumor antigen could induce autoimmune vitiligo, a response seen in approximately 36% of vaccinated mice (data not shown). To ensure the identity of the TRP2-specific memory T-cell population, we examined the CD8⁺ T cells for expression of CD127 (IL-7R), a marker shown to be selectively re-expressed upon differentiation of effector cells into memory cells. (21) Indeed, TRP2-specific CD8⁺ T cells isolated from spleen and lung expressed CD127 (FIG. 2F). Not only did the cells express CD127, but they remained fully functional, being able to produce both TNF and IL-2. Of the IFN⁺ cells found in lung and spleen, greater than 70% secreted TNF while greater than 20% secreted IL-2 (FIG. 2G). Furthermore, since a fraction of these cells acquired the ability to secrete IL-2 and express CD127, this indicates that this vaccination regimen generates memory cells of both effector and central memory phenotype. (22)

### Example 4

### In Contrast to CD40 Monotherapy, CD40/TLR7* Therapy Rescues CD8⁺ Memory T-Cell Function

Mice were immunized with 100 μg each of V peptide, CD40, and S-27609 in combinations as indicated. Memory CD8 functionality was assessed 65 days later. (A) Representative dot plots of IFN secretion by memory CD8⁺ T cells isolated from spleens and lungs of vaccinated mice. Dot plots are gated on live CD8⁺ cells, and numbers indicate the percentage of cells positive for both IFN and CD44. (B) Memory CD8⁺ T-cell cytolytic activity was assessed by performing an in vivo cytotoxicity assay. Numbers reflect the percentage of antigen-specific lysis. (C,D) Quantification of relative and absolute numbers of memory CD8 cells expressing IFN in the spleen (C) and lung (D). Absolute numbers of positive cells were determined by multiplying the relative percentage of each cell population by the total number of cells isolated from each tissue. (E) Quantification of the in vivo cytotoxicity assay presented in panel B. P.001 by one-tailed ANOVA. (F) CD127 expression on IFN⁺-memory CD8⁺ T cells derived from spleens or lungs of vaccinated mice. Isotype controls are shown as filled histograms. (G) Cytokine production by memory CD8⁺ T cells. Cells from panel F were analyzed for the ability to produce TNF and IL-2. Numbers reflect the percentage of CD8+IFN⁺ cells that also are positive for TNF or IL-2. In all cases, data are pooled from at least 2 independent experiments with 4 or more mice/group per experiment and plotted as means (±SEM).

### Example 5

### Superior Therapeutic Efficacy of CD40/TLR7* Immmunotherapy Compared with Either Monotherapy in Control of Metastatic Melanoma

The ability of different vaccination strategies to alter the progression of metastatic melanoma was compared. Mice were intravenously inoculated with 10⁵ metastatic B16.F10 melanoma cells and treatment was initiated 4 days later. Twenty-four days after vaccination, mice were killed and surface lung metastases were enumerated. Treatment with tumor antigen or tumor antigen plus a TLR7 agonist was ineffective in controlling tumor progression (FIG. 3A,B). Immunization with tumor antigen plus CD40 reduced the number of tumor nodules (P.001 vs V alone). However, addition of a TLR7 agonist to this vaccine resulted in a 3-fold reduction in the number of metastases over CD40 alone (FIG. 3B; P.01 vs V/CD40). Furthermore, the protection afforded by CD40/TLR7* relies upon antigen, as the removal of the H2K^{b} peptide, V, abrogates the effect of treatment (FIG. 3A,B). This protection is not unique to TLR7 agonists, as equal efficacy is observed with TLR3 and TLR9 agonists (data not shown). Moreover, changing the route of vaccination did not significantly alter the outcome of treatment (Figure S1, available on the Blood website; see the Supplemental Materials link at the top of the online article). Since CD40/TLR7* vaccination reduced the number of lung metastases, we asked whether combination immunotherapy would afford long-term protection against metastatic disease. All mice vaccinated with tumor antigen, tumor antigen plus TLR7 agonist, or CD40/TLR7 agonists without tumor antigen succumbed to lung failure (FIG. 3A). Mean survival times were 29, 30, and 30 days, respectively. CD40 monotherapy significantly increased survival times over tumor antigen alone (P.001) with a median survival time of 35 days and led to 3% of mice surviving greater than 90 days. However, the combination of tumor antigen plus CD40/TLR7* greatly improved survival over CD40 alone (P.001). Median survival times increased from 35 to 47 days with 20% of mice alive after 90 days (also see Kaplan-Meier plot in Figure S2). To determine which cellular subset mediates rejection of metastatic melanoma under this vaccination regimen, mice were depleted of CD8⁺, CD4⁺, and NK1.1⁺ cells prior to tumor challenge. Depletion of CD8S cells abrogated the protective effect of vaccination (FIG. 3C; P=0.001 compared with vaccination without depletion). Both CD4⁺ and NK1.1⁺ cells play a partial role in tumor protection, since their depletion resulted in slightly faster, although not significant, tumor progression (FIG. 3C). These data indicate that vaccination with combined immunotherapy, in the presence of antigen, leads to a CD8⁺ T cell-dependent immune response capable of mediating antitumor responses greater than that seen with either CD40- or TLR-based monotherapy.

### Example 6

### CD40/TLR7* Therapeutic Intervention Slows Progression of Metastatic Melanoma

C57BL/6 mice were challenged with 10⁵ metastatic B16.F10 melanoma cells intravenously. Four days later, mice were vaccinated with 100 μg of the tumor-associated antigen V, 100 μg CD40 FGK45, and 100 μg S-27609 in combinations as indicated. After 24 days, mice were killed, lungs were removed, and metastatic surface tumor nodules were enumerated with the aid of a dissecting microscope. (A) Photograph of macroscopically visible tumor nodules on lungs of mice, 24 days after tumor challenge. Numbers below the lungs reflect the mean survival time and long-term survival rate of mice monitored for therapeutic efficacy. Data are pooled from 3 to 4 independent experiments with greater than 8 mice per group in each experiment. (B) Enumeration of lung metastases. Data are pooled from 2 independent experiments and are presented as means plus or minus SEM (n=16 mice in each group). Data are representative of more than 4 separate experiments with at least 6 mice in each group. (C) Enumeration of lung metastases after effector cell depletion. Mice were treated as above except for the depletion of effector cell populations prior to tumor challenge as described in “Methods.” The data are expressed as means plus or minus SEM (n=8 mice in each group) and are representative of 3 independent experiments.

### Example 7

### Enhancement of Lung Infiltrates with Cytolytic Potential Following CD40/TLR7* Immunotherapy

To gain insight into why CD40/TLR7* immunotherapy mediated better antitumor immunity, we performed kinetic analyses of lung infiltrates 10 and 21 days after tumor challenge (FIG. 4A). Lymphocytes isolated from tumor-bearing lungs were subjected to intracellular cytokine staining after ex vivo peptide restimulation. Only tumor antigen plus either CD40 or CD40/TLR7* vaccination primed tumor-specific CD8⁺ T cells to migrate into the metastatic target organ (FIG. 4B). Flow cytometric analysis of V/CD40/TLR7* vaccinated mice revealed a 5-fold increase in the relative percentage of tumor-specific CD8⁺ T cells at day 10 and a 3-fold increase at day 21 over CD40 monotherapy. On an absolute scale, CD40 drives migration of polyclonal T cells into lungs of vaccinated mice irrespective of TLR stimulation, but this response wanes with time (FIG. 4C,D). In contrast, antigen-specific cells remain elevated, with CD40/TLR7* inducing greater absolute responses at both time points (P.001 between V/CD40/TLR7* and V/CD40 at both time points). Furthermore, cells generated from CD40/TLR7* vaccination showed cytolytic potential as measured by degranulation and Granzyme B expression (FIG. 4E).

### Example 8

### Kinetic Analysis of Lung-Infiltrating Lymphocytes

This example relates to experiments in FIG. 4. Shown in FIG. 4(A) is the experimental design, and FIG. 4(B) contains representative dot plots of lymphocytes isolated from metastatic target organs at day 10 or 21 after tumor challenge. Cells were isolated from tumor-bearing lungs as described in “Methods” and subjected to an in vitro restimulation with tumor peptide. Plots are gated on live, CD8⁺ cells. Numbers in the upper right-hand quadrant reflect the frequency of CD8⁺ T cells that are positive for both IFN and the activation marker CD44. Data are representative of 3 independent experiments with 4 mice per group in each experiment. (C,D) Quantification of lung infiltrates at either 10 (C) or 21 (D) days after tumor challenge. Data are plotted as means (±SEM) and represent pooled data from either 2 (C, n=8 mice/group) or 3 (D, n=12 mice/group) independent experiments, with 4 mice/group in each experiment. (E) Effector phenotype of CD8⁺ T cells isolated from lungs of mice vaccinated with tumor antigen plus CD40/TLR7* at either 10 or 21 days following tumor inoculation. The dot plots are first gated on live CD8⁺ cells and then further gated on IFN⁺CD44⁺ populations. Data are representative of at least 2 independent experiments, with 4 mice/group in each experiment.

### Example 9

Vaccine efficacy must overcome the effect of regulatory T cells, and the ratio of CD8⁺/FoxP3⁺ cells has been used to assess priming strength. (23) At day 10, combination therapy resulted in a 10-fold increase in the absolute numbers of antigen-specific CD8⁺ T cells to FoxP3⁺ cells, whereas CD40 monotherapy resulted in a 3-fold increase (FIG. 4C). We have shown that optimal reduction in the conversion of FoxP3⁻ “src=”/math/rarr.gif” border=0 FoxP3⁺ T cells requires the maturation of DCs with both CD40 and TLR agonists. These data support the hypothesis that one way in which combination immunotherapy mediates increased antitumor immunity is by amplifying CD8⁺ T-cell numbers and effector function while decreasing the effect of immunosuppression.

### Example 10

### CD40-Induced Hepatocellular Injury is Reduced by Coadministration of TLR7 Agonist

One of the significant dose-limiting safety concerns of the use of CD40 monotherapies is liver toxicity. Several human (24) and animal (24)^{\1 “B25”\“B26”} (27) studies using CD40 agonists report elevated levels of circulating hepatocyte enzymes ALT and AST, indicative of liver damage. To examine the severity of hepatocellular damage with monotherapy versus combination therapy, we measured plasma levels of ALT and AST in mice after vaccination (FIG. 5A,B). Both transaminases were significantly elevated in mice treated with CD40, peaking at 48 hours after treatment. TLR7* had no effect on enzyme levels. In contrast to CD40 monotherapy, CD40/TLR7* treatment completely ameliorated the toxicity seen with CD40 alone. Macroscopic evaluation of livers revealed substantial areas of necrosis, a finding observed only in mice treated with CD40 (data not shown). Histologic analysis confirmed the severity of hepatocellular damage (FIG. 5C-F). Normal liver architecture was seen in mice treated with PBS (FIG. 5C). Livers isolated from mice treated with CD40 exhibited widespread bridging coagulative necrosis (FIG. 5D), whereas TLR7* treatment resulted in minor inflammation without any observable coagulative necrosis (FIG. 5E). Livers from mice receiving CD40/TLR7* had some foci of inflammation but little to no coagulative necrosis (FIG. 5F). The extent of histologic damage was subsequently scored on a semiquantitative scale (FIG. 5G). The data revealed that TLR7* significantly reduces liver toxicity associated with CD40 monotherapy (P=0.026). Although it is not clear why TLR7* attenuates CD40-induced toxicity, we have shown that this reversion in toxicity is TLR7 dependent, as both MyD88 KO and TLR7 KO mice had similar ALT and AST enzyme levels when treated with either CD40 or CD40/TLR7* (data not shown). Finally, whereas the molecular and cellular mechanism for CD40/TLR7* combination therapy in reversing toxicity remains unclear and requires further investigation, it nonetheless not only provides better therapeutic outcomes but also minimizes adverse side effects.

### Example 11

### Hepatic Toxicity Associated with CD40 Monotherapy is Reversed with TLR7 Agonism

This example relates to experiments in FIG. 5. FIG. 5(A, B) contain kinetic analysis of serum transaminases. Mice were treated with PBS, 100 μg CD40, 100 g TLR7*, or both intravenously. Serum was isolated at various time points afterward, and serum levels of alanine transaminase (A) or aspartate transaminase (B) were measured as described. Data are representative of 3 independent experiments, with n=3 to 8 mice per group, per time point. (C—F) Histologic analysis of livers treated with PBS (C), 100 μg CD40 (D), 100 μg TLR7*(E), or 100 μg CD40 and 100 μg TLR7*(F) for 48 hours. (G) Semiquantitative assessment of histopathologic changes in livers from mice treated as above for 48 hours. Data are pooled from 2 independent experiments, with n=6 mice in each treatment group. P=0.026 by Mann-Whitney nonparametric test.

### Example 12

### Abatement of Liver Toxicity by Co-Administration of TLR Agonist or IFNa with a CD40 Agonist

This example relates to the experiments in FIGS. 6 and 7. Therein hepatocellular injury was biochemically assessed by measuring liver enzyme activity. Specifically mice received 100 mg anti-CD40, 100 mg S-27609 or both IV. In some cases mice also received graded doses of recombinant interferon alpha (normally, one million international units per mouse). Serum was harvested 24-72 hours later and sent to Charles River Laboratories (worcester, Mass.) for liver chemistry profile analysis. Alternatively, serum samples were anallyzed by the National Jewish Medical Center ore Lab (Denver, Colo.)

### CONCLUSIONS

While the past 10 years have witnessed an exponential growth in the identification of cancer target antigens, a similar pace for the development of human adjuvants to effectively immunize against these targets has lagged. The molecular identification of Toll-like Receptors and their ligands, and receptor-ligands that control adaptive immunity have provided the first logical, hypothesis-based strategies to molecularly concoct adjuvants so as to elicit protective immune responses to cancer. Parallel to the importance of TLRs in mobilizing the innate immune response, CD40 and its ligand are the central activators for the development of the adaptive immune responses. Our data show that the use of well-defined agonists that activate specific TLRs, combined with the use of agonists for CD40, elicit profound cell-mediated immune responses to defined peptides that meet or exceed that which is seen with the most potent viral vectors. Based on these observations, we have used this CD40/TLR platform and have shown that it can be therapeutically effective in the treatment of melanoma. We hypothesize that these two agonists impinge on the dendritic cell (DC) as a target, and induce functional features which uniquely empower the DC to drive profound CMI responses. While we do not fully understand why these DCs are so effective in inducing CMI, we show that the molecular signature of DCs triggered with TLR* and DCD40 is distinctive from DCs triggered with either agent alone in vivo.

Perhaps one of the weakest aspects of our approach to fight cancer, is the lack of adjuvants that can elicit robust, long-lasting immunity to cancer-related antigens. In the past, we have relied on the use of agents that appeared to induce inflammation. Alum is salts of aluminum hydroxide and phosphate and primarily elicits humoral-mediated immune responses. This adjuvant was first employed in 1926 and was effectively grandfathered in when the FDA first assumed new drug approval authority in 1938. Alum is the only FDA approved adjuvant, and is a component of a number of our commonly used vaccines, like tentanus toxoid. There are many other adjuvants (non-cytokine) that have been employed in cancer clinical trials like Bacille Calmette-Guérin (BCG), keyhole limpet hemocyanin (KLH), incomplete Freund's adjuvant (IFA), all which have poorly understood mechanisms of action and modest adjuvant activities. Not until 1999, when the first studies elucidating the receptors for immune adjuvants (Toll-like receptors) emerged on the horizon, did a molecular understanding of how these “non-specific” activators of the immune system trigger innate immunity.

TLRs are type 1 membrane proteins that are expressed on hematopoietic and non-hematopoietic cells. Currently, there are 11 members in the TLR family. These receptors are characterized by their capacity to recognize pathogen-associated molecular patterns (PAMP) expressed by pathogenic organisms. Typical PAMPS include LPS, DNA (CpG), lipoproteins, ssRNA, and glycolipids, as detailed in the Table I below. Whether there are true endogenous ligands for TLRs is still controversial, although it has been reported that TLR2 and TLR4 are able to recognize several self-proteins including members of heat shock protein family hsp60 and hsp70.

In general, triggering of TLR elicits profound inflammatory responses through enhanced cytokine production (IL12, IL18, etc), chemokine receptor expression (CCR2, CCR5 and CCR7), and costirnmulatory molecule expression. As such, these receptors in the innate immune systems exert control over the polarity of the ensuing acquired immune response.

CD154, the ligand for CD40 (CD40L, gp39) is a 32-39 kD member of the Tumor Necrosis Factor Family, which includes TNF-α, lymphotoxin, FasL, CD30L, CD27L, 4-1BBL, and OX-40L. Activated CD4 T-cells are the predominant cell type responsible for CD154 expression. Expression of CD154 on CD8⁺ T-cells, eosinophils, mast cells and basophils, NK cells, and DCs has also been described. The receptor for CD154, CD40 is a member of the tumor necrosis factor receptor (TNF-R) superfamily that includes TNF-RI (p55), TNF-RII (p75), p75 neurotrophin receptor, fas, CD30, CD27, 4-1BB, and OX-40. It is a 50-kDa membrane protein whose tissue distribution was originally thought to be restricted to B cells, DCs (DC's) and basal epithelial cells however, later studies have shown functional expression of CD40 on monocytes/macrophages, microglial cells and endothelial cells.

In vitro studies on isolated DCs have shown that CD40 triggering alters the expression of cytokines (IL12, IL15), chemokines (IP10, MIP-1alpha MIP-1beta and IL-8) co-stimulatory molecule expression (CD80, CD86) and chemokine receptors All of these effects culminate in the ability of CD40-activated DCs to stimulate enhanced T cell proliferation and differentiation. Our own data shows that CD154 exerts far more profound effects on the early signaling, cytokine production and chemokine production compared to TNF□ and RANKL. One other critical impact of CD40 triggering of DCs is the change in the turnover of peptide-MHCII. Lanzavecchia has shown using LPS and we have shown using sCD154 that maturation of DCs with a CD40 agonist facilitates the accumulation of MHCII-peptide complexes on the surface of DCs. Studies from our lab and others indicate that CD40 appears to be a critical longevity signal for DCs in vivo. We have hypothesized that DC longevity is essential for the prolonged clonal expansion of CD4⁺ T cell responses. The impact of CD40 signaling on DC longevity, we feel is a critical feature of the synergy that is observed when TLR and CD40 agonists are used in combination and will be discussed below. In summary, there is no doubt that CD40 agonists induce profound biologic changes in DCs in vitro and in vivo. However, we hypothesize that these changes are not sustained, ineffective and inadequate to “license” the DC to truly trigger effective CMI responses.

The success of CD40 agonists to elicit CMI in the absence of CD4⁺ T cells generated substantial enthusiasm to use CD40 agonists as adjuvants for cancer vaccines. A series of studies by Glennie and co-workers showed that one can achieve tumor regression of CD40⁺ lymphoma using □CD40, but the doses of CD40 agonist were very high (250 ug/day, days 2-5), and oddly, the tumor inoculum needed for immunization was very high (5×10⁷/mouse). Nonetheless, clinical remission of these CD40⁺ lymphoma was impressive. Less impressive were studies on hematopoietic tumors which were CD40⁻. It is likely that the successes with CD40⁺ lymphomas and leukemias were due to direct effects of CD40 agonists on the tumor. For lymphomas and leukemias, □CD40 may also enhance their APC activities, and at the same time enhance their apoptosis. Later studies by this same group, however, did demonstrate that CD40 agonists could exert beneficial therapeutic effects on solid tumors. With solid tumors, a number of studies have shown that CD40 activation promotes apoptotic death and that CD40 expression is an important factor in the generation of tumor-specific T-cell responses that contribute to tumor cell elimination. Other groups, like that of Melief and co-workers have shown that CD40 agonists alone or TLR agonists alone could elicit effective therapeutic on Ad5E1A expressing (CD40−) tumors in vivo (tumor type not described). Using a renal cell carcinoma model, Murphy and co-workers have shown that only the combination of an agonist D CD40 and IL-2, but neither agent administered alone, induced complete regression of metastatic tumor and specific immunity to subsequent rechallenge in the majority of treated mice. At this time efficacy with CD40 agonists alone is unpredictable. It is not clear if CD40 expression on the tumor is important, if tumor burden is important, if CD40 alone is adequate and if there is a distinctive difference in the efficacy of CD40 therapy in liquid or solid tumors. We would contend that when used with a TLR agonist, CD40 agonists will induce high levels of tumor-specific immunity, and avoid the idiosyncrasies seen in different tumor models with □CD40 monotherapy.

CD40 is a reasonable target for inducing heightened CMI responses for the purposes of tumor protection, yet the data in the literature suggested that it was not applicable in a wide range of tumors. My laboratory has worked intensively for a number of years to try to develop a general method to enhance protective tumor immunity using □CD40 as a monotherapy, and failed. Any and all parameters of dose of antibody, timing, route of inoculation, tumor type, different mabs, etc were extensively tested yet these efforts proved futile, except in B lymphoma and leukemia models, as reported by Glennie. A recent study from Kedl and co-workers has shed much light on some of the important parameters that may influence the generation of protective CTL when using CD40 agonists. Using tetramer staining for SIINFYKL-specific CTL, and OVA-transduced B16, they showed that Q CD40 agonists actually accelerated the loss of SIINFYKL-specific CTL. However, if immunization were done with a vaccinia virus carrying a SIINFYKL minigene, enhanced CTL expansion was observed using □CD40 agonists. It was concluded that long-term immunization to tumor antigens are only enhanced by CD40 agonists if those tumor antigens are delivered in viral vectors or in the context of inflammation. Hence, the great disparities in the outcome of innumerable tumor models may be due to the inadvertent addition of co-inflammatory mediators that synergize with the □CD40 agonist.

Such in vivo studies led to a number of recent reports on the requirements of co-signals for the activation of DCs by CD40 agonists. Published studies, as well as those to be presented in the Preliminary Data section, show that CD40 engagement alone is insufficient to induce IL12p70 production by DCs in vitro and in vivo. By evaluating mRNA for p40 and p35, the authors show that co-engagement via TLR (STAg, an extract from Toxoplasma gondi) and CD40 is critical for enhanced p35 mRNA expression and the production of IL12p70. This study was followed by an investigation using human DCs where it was shown that CpG DNA was a critical co-stimulus with CD40 signaling for IL12p70 production in vitro (51). Taken together, these were the first studies to document that CD40 was necessary but not sufficient to drive DC certain aspects of DC maturation. However, they did not provide compelling evidence that the combined actions of CD40 and TLR agonism was essential to fulminately elicit CML

The question of synergy between CD40 and TLR agonism was approached directly by quantifying the impact of either TLR or CD40 engagement or TLR/CD40 engagement on the expansion of OVA-specific tetramer⁺ cells in vivo. We have shown that the administration of □CD40, a TLR7 agonist (S27609) and OVA (protein or peptide) can induce the generation of OVA-specific CD8⁺ T cells (see examples in Preliminary data section). By day 6, the antigen-specific T cells can represent over 25% of the entire CD8⁺ T cell population. All TLR agonists tested synergize with anti-CD40 and induce potent antigen-specific CTL activities. These findings supported the hypothesis that the combined triggering of innate and acquired immunity maximized the capacity to induce potent effector T cells and set the stage for the use of this technology as a vaccine platform in cancer immunotherapy.

Studies in both mouse and human have shown that the administration of CD40 agonists alone induce toxicity. In intact mice, it has been shown that CD40 agonists induce liver toxicity. In immune deficient mice and non-lethally-irradiated mice, the administration of CD40 agonists induce lethality. During the course of our studies with combined administration of □CD40 and TLR agonists (or IFNa) we discovered that the addition of either a TLR agonist or IFNa in vivo to mice treated with □CD40 resolved toxicity. Thus the co-administration of IFNa or TLR agonist with a CD40 agonist should resolve the toxicity observed in the clinical use of CD40 agonists.

In addition, the identification of molecular triggers for innate and adaptive immunity will revolutionize adjuvant platforms for vaccines. However, isolated activation of one immune pathway in the absence of others may be toxic, ineffective, and in some cases detrimental to the development of long-term, protective immunity. More effective molecularly engineered vaccines will likely include combinations of agents that trigger multiple immunologic pathways. (28, 29) Our studies demonstrate that CD40 and TLR agonists in combination, compared with either unitary adjuvant, elicit (1) high frequencies of self-reactive, effector CD8⁺ T cells, (2) potent, tumor-specific CD8⁺ memory, (3) CD8⁺ T cells that efficiently infiltrate metastatic target organs and exert effector functions, (4) superior therapeutic efficacy, (5) heightened ratios of CD8⁺ T cells to FoxP3⁺ T cells at the tumor site, and (6) reduced hepatotoxicity.

Heightened frequencies of tumor-specific CD8⁺ T cells have been primary end points for many human clinical trials (13) and are believed to be a necessary component in the emergence of protective antitumor immunity. The frequency of antigen-specific CD8⁺ T cells that are elicited by the combined administration of CD40/TLR agonist and antigen is an order of magnitude higher than that observed with almost any other adjuvant or cell-based vaccine platform, such as antigen-pulsed DCs.(30)^{\1 “B31”-} (32) While the cellular and molecular basis for this striking response is incompletely understood, we have published that the expression of CD70 on DCs is critical for CD8⁺ T-cell expansion. (9) Heightened expression of CD70 on CD8⁻ DCs is induced only when both CD40 and TLR agonists are coadministered. The subsequent increased signaling through CD70/CD27 could account for the superior memory responses seen after vaccination. (33) Other data suggests that CD8⁻ DCs acquire the capacity to cross-present soluble antigen when triggered via CD40 and TLR in vivo, and this too may contribute to the extremely high frequencies of antigen-specific CD8⁺ T cells. Overall, our current hypothesis is that CD40/TLR7* increases the efficiency of antigen processing and cross-presentation thereby facilitating enhanced CD8⁺ T-cell priming and memory. The data presented herein used peptide antigen, and as such, bypassed the cross-presentation pathway. However, it is interesting to speculate that CD40/TLR agonism may facilitate epitope spreading to alternative tumor antigens after peptide vaccination

Anti-CD40 as a unitary adjuvant has been shown to terminate both humoral (34) and cell-mediated immune (16) responses. While CD40 monotherapy may provide a minimal enhancement of short-term immunity, studies have shown that it abbreviates the generation of CD8⁺ T-cell memory. (14) Interestingly, even for humoral immunity, the use of CD40 agonists aborts long-term memory and the generation of long-lived plasma cells. (17) In recent studies by Murphy and coworkers (Berner et al (14)), CD40 monotherapy resulted in the IFN-dependent apoptosis of tumor-specific CD4⁺ T cells and the inability to mount protective memory responses to tumor challenge. A number of CD40 monoclonal antibodies have entered the clinic, (2, 4, 35)^{1 “B36”1 “B37”-} (38) only one (2) of which has been reported to be a strong agonist, similar to the antimurine CD40 used herein and in a wealth of other murine studies, for example. (39, 40) In that phase 1 study, 4 patients, each with stage IV melanoma, were found to have a partial response on restaging at the end of study. While it may be premature to make any conclusive statements concerning agonistic CD40 monotherapy (2) as a vaccine platform, the preclinical studies in mice certainly suggest that it would be more effective as a vaccine when combined with activators of innate immunity. Even if not for clinical efficacy, the toxicity of CD40 monotherapy may be ameliorated with the addition of other immune activators. One indication where agonistic CD40 monotherapy may be suitable is in B-cell lymphoma where, in mice, high-dose monotherapy has been shown to be extremely effective (40, 41). Studies in animal models reveal that as unitary adjuvants, TLR agonists can elicit robust, inflammatory responses and enhance a wide spectrum of specific immune responses. (42)

Results of clinical studies with TLR agonists have been mixed (43) Imiquimod, an FDA-approved topically applied TLR7 agonist, has proven extremely effective in basal cell carcinoma. Furthermore, 2 improved adult hepatitis B virus (HBV) vaccines using TLR4 agonists have been approved. However, in June 2007, Pfizer suspended a clinical program in non-small cell lung cancer for a TLR9 agonist due to lack of clinical efficacy in phase 2 and 3 trials when combined with a variety of chemotherapeutic agents. (44) Our data strongly suggest that, at least in cancer indications, activators of adaptive immunity will greatly augment the therapeutic potential of TLR agonists.

It is encouraging that single-arm trials with TNFR agonists and TLR agonists have been shown to be largely safe and induce inflammatory responses. Based on emerging preclinical studies in mice using admixtures of TLR agonists, TNFR agonists, and other immune activators, it is anticipated that these admixtures will greatly improve efficacy in clinical trials, and at the same time reduce toxicity. Enhanced frequencies of primary effector T cells, potent long-term immunologic memory, and reduced regulatory T-cell functions are some of the hallmark end points that likely need to be achieved for successful therapeutic intervention. The findings of this and other studies (45) provide rational strategies for the creation of multifactorial vaccines to achieve maximal efficacy in cancer vaccine trials in humans.

### LIST OF REFERENCES

1. CpG 7909: PF 3512676, PF-3512676. Drugs R D 2006; 7:312-316.
2. Vonderheide R H, Flaherty K T, Khalil M, et al. Clinical activity and immune modulation in cancer patients treated with CP-870,893, a novel CD40 agonist monoclonal antibody. J Clin Oncol 2007; 25:876-883.
3. Younes A. CD40 ligand therapy of lymphoma patients. J Clin Oncol 2001; 19:4351-4353.
4. Vonderheide R H, Dutcher J P, Anderson J E, et al. Phase I study of recombinant human CD40 ligand in cancer patients. J Clin Oncol 2001; 19:3280-3287.
5. Janeway C A Jr and Medzhitov R. Innate immune recognition. Annu Rev Immunol 2002; 20:197-216.
6. Kopp E and Medzhitov R. Recognition of microbial infection by Toll-like receptors. Curr Opin Immunol 2003; 15:396-401.
7. Heath W R, Belz G T, Behrens G M, et al. Cross-presentation, dendritic cell subsets, and the generation of immunity to cellular antigens. Immunol Rev 2004; 199:9-26.
8. Ahonen C L, Doxsee C L, McGurran S M, et al. Combined TLR and CD40 triggering induces potent CD8+ T cell expansion with variable dependence on type I IFN. J Exp Med 2004; 199:775-784.
9. Sanchez P J, McWilliams J A, Haluszczak C, Yagita H, Kedl R M. Combined TLR/CD40 stimulation mediates potent cellular immunity by regulating dendritic cell expression of CD70 in vivo. J Immunol 2007; 178:1564-1572
10. van Duin D, Medzhitov R, Shaw A C. Triggering TLR signaling in vaccination. Trends Immunol 2006; 27:49-55.
11. Heckelsmiller K, Beck S, Rall K, et al. Combined dendritic cell- and CpG oligonucleotide-based immune therapy cures large murine tumors that resist chemotherapy. Eur J Immunol 2002; 32:3235-3245.
12. Kochling J, Konig-Merediz S A, Stripecke R, et al. Protection of mice against Philadelphia chromosome-positive acute lymphoblastic leukemia by cell-based vaccination using nonviral, minimalistic expression vectors and immunomodulatory oligonucleotides. Clin Cancer Res 2003; 9:3142-3149.
13. Speiser D E, Lienard D, Rufer N, et al. Rapid and strong human CD8+ T cell responses to vaccination with peptide, IFA, and CpG oligodeoxynucleotide 7909. J Clin Invest 2005; 115:739-746.
14. Berner V, Liu H, Zhou Q, et al. IFN-gamma mediates CD4+ T-cell loss and impairs secondary antitumor responses after successful initial immunotherapy. Nat Med 2007; 13:354-360
15. Bartholdy C, Kauffmann S O, Christensen J P, Thomsen A R. Agonistic anti-CD40 antibody profoundly suppresses the immune response to infection with lymphocytic choriomeningitis virus. J Immunol 2007; 178:1662-1670.
16. Kedl R M, Jordan M, Potter T, Kappler J, Marrack P, Dow S. CD40 stimulation accelerates deletion of tumor-specific CD8(+) T cells in the absence of tumor-antigen vaccination. Proc Natl Acad Sci USA 2001; 98:10811-10816.
17. McWilliams J A, McGurran S M, Dow S W, Slansky J E, Kedl R M. A modified tyrosinase-related protein 2 epitope generates high-affinity tumor-specific T cells but does not mediate therapeutic efficacy in an intradermal tumor model. J Immunol 2006; 177:155-161
18. Betts M R, Brenchley J M, Price D A, et al. Sensitive and viable identification of antigen-specific CD8+ T cells by a flow cytometric assay for degranulation. J Immunol Methods 2003; 281:65-78.
19. Burkett M W, Shafer-Weaver K A, Strobl S, Baseler M, Malyguine A. A novel flow cytometric assay for evaluating cell-mediated cytotoxicity. J Immunother (1997) 2005; 28:396-402.
20. Rubio V, Stuge T B, Singh N, et al. Ex vivo identification, isolation and analysis of tumor-cytolytic T cells. Nat Med 2003; 9:1377-1382.
21. Kaech S M, Tan J T, Wherry E J, Konieczny B T, Surh C D, Ahmed R. Selective expression of the interleukin 7 receptor identifies effector CD8⁺ T cells that give rise to long-lived memory cells. Nat Immunol 2003; 4:1191-1198.
22. Wherry E J, Barber D L, Kaech S M, Blattman J N, Ahmed R. Antigen-independent memory CD8⁺ T cells do not develop during chronic viral infection. Proc Natl Acad Sci USA 2004; 101:16004-16009.
23. Quezada S A, Peggs K S, Curran M A, Allison J P. CTLA4 blockade and GM-CSF combination immunotherapy alters the intratumor balance of effector and regulatory T cells. Clin Invest 2006; 116:1935-1945
24. Zhou F, Ajuebor M N, Beck P L, Le T, Hogaboam C M, Swain M G. CD154-CD40 interactions drive hepatocyte apoptosis in murine fulminant hepatitis. Hepatology 2005; 42:372-380.
25. Kimura K, Moriwaki H, Nagaki M, et al. Pathogenic role of B cells in anti-CD40-induced necroinflammatory liver disease. Am J Pathol 2006; 168:786-795.
26. Kimura K, Kakimi K, Wieland S, Guidotti L G, Chisari F V. Activated intrahepatic antigen-presenting cells inhibit hepatitis B virus replication in the liver of transgenic mice. J Immunol 2002; 169:5188-5195.
27. Schmitz V, Dombrowski F, Prieto J, et al. Induction of murine liver damage by overexpression of CD40 ligand provides an experimental model to study fulminant hepatic failure. Hepatology 2006; 44:430-439.
28. Pure E, Allison J P, Schreiber R D. Breaking down the barriers to cancer immunotherapy. Nat Immunol 2005; 6:1207-1210.
29. Kornbluth R S and Stone G W. Immunostimulatory combinations: designing the next generation of vaccine adjuvants. J Leukoc Biol 2006; 80:1084-1102.
30. Fay J W, Palucka A K, Paczesny S, et al. Long-term outcomes in patients with metastatic melanoma vaccinated with melanoma peptide-pulsed CD34(+) progenitor-derived dendritic cells. Cancer Immunol Immunother 2006; 55:1209-1218.
31. Lesimple T, Neidhard E M, Vignard V, et al. Immunologic and clinical effects of injecting mature peptide-loaded dendritic cells by intralymphatic and intranodal routes in metastatic melanoma patients. Clin Cancer Res 2006; 12:7380-7388
32. Mackensen A, Meidenbauer N, Vogl S, Laumer M, Berger J, Andreesen R. Phase I study of adoptive T-cell therapy using antigen-specific CD8+ T cells for the treatment of patients with metastatic melanoma. J Clin Oncol 2006; 24:5060-5069.
33. Borst J, Hendriks J, Xiao Y. CD27 and CD70 in T cell and B cell activation. Curr Opin Immunol 2005; 17:275-281.
34. Erickson L D, Durell B G, Vogel L A, et al. CD40 agonists short circuit humoral immunity. J Clin Invest 2002; 109:613-620.
35. Vonderheide R H. Prospect of targeting the CD40 pathway for cancer therapy. Clin Cancer Res 2007; 13:1083-1088.
36. Geldart T R, Harvey M, Carr N, Glennie M, Johnson P. Cancer immunotherapy with a chimeric anti-CD40 monoclonal antibody: evidence of preclinical efficacy [abstract]. J Clin Oncol 2004; 22:2577.
37. Fanale M A and Younes A. Monoclonal antibodies in the treatment of non-Hodgkin's lymphoma. Drugs 2007; 67:333-350
38. Kelley S K, Gelzleichter T, Xie D, et al. Preclinical pharmacokinetics, pharmacodynamics, and activity of a humanized anti-CD40 antibody (SGN-40) in rodents and non-human primates. Br J Pharmacol 2006; 148:1116-1123.
39. Mauri C, Mars L T, Londei M. Therapeutic activity of agonistic monoclonal antibodies against CD40 in a chronic autoimmune inflammatory process. Nat Med 2000; 6:673-679.
40. French R R, Chan H T, Tutt A L, Glennie M J. CD40 antibody evokes a cytotoxic T-cell response that eradicates lymphoma and bypasses T-cell help. Nat Med 1999; 5:548-553.
41. Sotomayor E M, Borrello I, Tubb E, et al. Conversion of tumor-specific CD4+ T-cell tolerance to T-cell priming through in vivo ligation of CD40. Nat Med 1999; 5:780-787.
42. Krieg A M. CpG motifs in bacterial DNA and their immune effects. Annu Rev Immunol 2002; 20:709-760
43. Kanzler H, Barrat F J, Hessel E M, Coffman R L. Therapeutic targeting of innate immunity with Toll-like receptor agonists and antagonists. Nat Med 2007; 13:552-559.
44. Schmidt C. Clinical setbacks for toll-like receptor 9 agonists in cancer. Nat Biotechnol 2007; 25:825-826.
45. Uno T, Takeda K, Kojima Y, et al. Eradication of established tumors in mice by a combination antibody-based therapy. Nat Med 2006; 12:693-698.

It is understood that modifications which do not substantially affect the activity of the various embodiments of this invention are also provided within the definition of the invention provided herein.

The various references to journals, patents, and other publications which are cited herein comprise the state of the art and are incorporated by reference as though fully set forth.

1. An improved therapeutic regimen that involves the administration of at least one TNF-R agonist at a dosage that elicits liver toxicity in some subjects when administered as a monotherapy wherein the improvement comprises further administering an amount of at least one type 1 interferon and/or TLR agonist sufficient to eliminate or reduce said liver toxicity by at least 50% as determined based on liver enzyme levels.

2. The regimen of claim 1 wherein the TNF-R agonist is a CD40 agonist.

3. The regimen of claim 2 wherein the CD40 agonist is an agonistic antibody or fragment or a monomeric or multimeric CD40L polypeptide or variant or fragment or conjugate having CD40 agonistic activity.

4. The regimen of claim 1, wherein the TLR agonist is an agonist of a TLR selected from the group consisting of TLR1, TLR2, TLR3, TLR4, TLR5, TLR6, TLR7, TLR8, TLR9, TLR10, TLR11, and TLR12.

5. The regimen of claim 1 wherein the TLR agonist is a yeast or bacterial spheroplast, cytoplast, membrane, or subcellular particle.

6. The regimen of claim 1 wherein the TNF-R agonist is a CD40 agonist that is administered at a dosage at least 2 times the amount that elicits liver toxicity as a monotherapy.

7. The regimen of claim 1 wherein the TNF-R agonist is a CD40 agonist that is administered at a dosage at least 5 times the amount that elicits liver toxicity as a monotherapy.

8. The regimen of claim 1 wherein the TNF-R agonist is a CD40 agonist that is administered at a dosage at least 10 times the amount that elicits liver toxicity as a monotherapy.

9. The regimen of claim 1 that further comprises administering an antigen to which an immune response is to be elicited.

10. The regimen of claim 9 wherein said antigen is a viral, bacterial, fungal, or parasitic antigen.

11. The regimen of claim 9 wherein said antigen is a human antigen.

12. The regimen of claim 11 wherein said human antigen is a cancer antigen, autoantigen or other human antigen the expression of which correlates or is involved in a chronic human disease.

13.-17. (canceled)

18. The regimen of claim 9 wherein the antigen is an autoantigen the expression of which correlates to an autoimmune disease.

19. A regimen of claim 1 that elicits an antigen specific cellular immune response.

20. The regimen of claim 19, wherein said administering results in a least one of the following:
(i) enhanced primary and memory CD8+ T cell responses relative to the administration of a DNA encoding only a CD40 agonist or TLR agonist or type 1 interferon;
(ii) induces exponential expansion of antigen specific CD8+ T cells; and
(iii) generates a protective immune response in a CD4 deficient host comparable to a normal (non-CD4 deficient) host.

21. The regimen of claim 1, which is used to treat a disease selected from the group consisting of cancer, allergy, inflammatory disease, infectious disease and an autoimmune disease.

22. The regimen of claim 21 wherein the infectious disease is caused by a virus, bacterium, fungus, or parasite and the TLR agonist comprises the virus, bacterium, fungi, or parasite or fragment or portion thereof that causes the disease or a virus or microorganism engineered to express an antigen thereof.

23. The regimen of claim 22 wherein the virus is HIV.

24. The regimen of claim 1 which is used to treat melanoma, lung cancer, lymphoma or leukemia.

25.-26. (canceled)

27. The regimen of claim 24, wherein the lymphoma or leukemia is a B cell lymphoma or CLL
